(19) United States
(12) Patent Application Publication
(10) Pub. No.: US 2015/0132271 A1
(43) Pub. Date: May 14, 2015

(54) READY TO EAT COLD BEVERAGE CAKES WITH INGREDIENTS

(71) Applicant: Alice Chang, South Pasadena, CA (US)

(72) Inventor: Alice Chang, South Pasadena, CA (US)

(21) Appl. No.: 14/078,394

(22) Filed: Nov. 12, 2013

(51) Int. Cl. A23L 1/054; A61K 31/4709; A61K 35/64; A61K 38/47; A61K 36/06

(52) U.S. Cl. CPC A23L 1/0545; A61K 38/47; A61K 36/06; A61K 35/644; A61K 31/4709; C12Y 302/01108
USPC 424/93.51; 426/573; 426/576; 426/578; 426/89; 426/72; 426/103; 426/574; 424/94.61; 424/539; 514/305

(57) ABSTRACT

Cold and ready to eat beverage cakes, and with a variety of ingredient combinations are suitable for healthy consumption. The beverage cakes are soft in texture and high in nutrition. People can save the time and energy of preparing and cooking of the food. Serving size can be a single serving, and a six pack of servings. Beverage cakes are also created as beverage egg cakes, beverage plus salt source cakes, beverage seafood cakes, beverage meat cakes, beverage bubbles, beverage balls, beverage patties, and beverage sausages. They can be served as cold and hot food for breakfast, lunch, snack, and dinner. They can also be used as animal food, baby food, for children with less teeth, for elderly people, and for sick people. Cold and ready to eat beverage cakes with ingredients offer the consumers an easy and convenient way to obtain an affordable, nutritious, and delicious meal.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR 1.71(d).

### BACKGROUND OF THE INVENTION

The following includes information that may be useful in understanding the present invention(s). It is not an admission that any of the information provided herein is prior art, or material, to the presently described or claimed inventions, or that any publication or document that is specifically or implicitly referenced is prior art.

1. Field of the Invention

The present invention relates generally to the field of comestibles and more specifically relates to ready to eat cold beverage cakes with ingredients.

The beverage is created as beverage cakes in various shapes and sizes. And the beverage cake is created as cake made with protein and/or starch. Gelatin from fish, animal, and egg is used as the protein which is collagen essential to the human body. Vegetable gum and vegetable starch are used as the carbohydrate which is also essential to the human body so that the consumer can consume the beverage in the form of cake, with essential nutrition of collagen and carbohydrates.

2. Description of the Related Art

Beverage is a kind of liquid which is specifically prepared for human consumption. The beverage can be divided into various groups such as plain water, alcohol, non-alcoholic drink, soft drink, fruit juice, vegetable juice, tea coffee, milk, and hot drink. Beverage is typically created as a liquid medium, and not as ready to eat form. Nutrition, if maintained, may help individuals avoid serious health problems.

Various attempts have been made to solve the above-mentioned problems such as those found in U.S. Pat. and Pub. Nos. 2013/0156894; U.S. Pat. Nos. 8,277,858; 5,747,087; 8,192,784; 4,059,046; and 5,264,238. This prior art is representative of comestibles relating to food and beverage compositions. None of the above inventions and patents, taken either singly or in combination, is seen to describe the invention as claimed.

Ideally, ready to eat cold beverage cakes with ingredients should provide nutrition and convenience to consumers and, yet would be manufactured at a modest expense. Thus, a need exists for ready to eat cold beverage cakes with ingredients.

### BRIEF SUMMARY OF THE INVENTION

In view of the foregoing disadvantages inherent in the known beverages related comestibles art, the present invention provides novel ready to eat cold beverage cakes with ingredients. The general purpose of the present invention, which will be described subsequently in greater detail is to provide ready to eat cold beverage cakes with a variety of ingredients. The invention is to provide people who shop for food the option of getting processed and cooked beverage cakes, cold and ready to eat.

A ready to eat cold beverage cake product comprising: beverage cake, having at least one beverage ingredient, and at least one binding agent. Flavorings and other ingredients cause the beverage cakes to be flavored, as per consumer preference, for consumption. Various flavored versions of the beverage cakes may be produced. Kits may be packaged with ingredients and sold for home cooking.

A method of preparing ready to eat cold beverage cakes with ingredients comprises the steps of: stifling and mixing at least one beverage with at least one binding agent; boiling until texture of admixture is soft and smooth; adding ingredient and flavoring as an admixture; forming beverage cake; storing beverage cake in a refrigerator to congeal and solidify; and optionally packaging to preserve freshness once solidified.

The present invention holds significant improvements and serves as a ready to eat cold beverage cakes system. For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and method(s) of use for the present invention, ready to eat cold beverage cakes constructed and operative according to the teachings of the present invention.

FIG. 1 shows a perspective view illustrating a square packaging option for ready to eat cold beverage cakes according to an embodiment of the present invention.

FIG. 2 is a perspective view illustrating a six-pack of individually packed ready to eat cold beverage cakes portions ready to consume according to an embodiment of the present invention of FIG. 1.

FIG. 3 is a perspective view illustrating small spheres or bubbles of ready to eat cold beverage cakes that may be mixed into drinks, food, and condiments according to an embodiment of the present invention of FIGS. 1 & 2.

FIG. 4 is a perspective view illustrating ready to eat cold beverage cakes portions in the form of balls, patty, and sausage according to an embodiment of the present invention of FIG. 1.

FIG. 5 is a flowchart illustrating a method of preparing the ready to eat cold beverage cakes according to an embodiment of the present invention of FIGS. 1-4.

The various embodiments of the present invention will hereinafter be described in conjunction with the appended drawings, wherein like designations denote like elements.

### DETAILED DESCRIPTION

As discussed above, embodiments of the present invention relate to a comestible and more particularly to ready to eat cold beverage cakes with ingredients as used to improve the health of consumers. The purpose as previously mentioned is to create healthy comestibles by creating novel ready to eat cold beverage cakes, and with a variety of ingredients. Cold and ready to eat beverage cakes with ingredients save people the time and energy of preparing and cooking of the food. Cold and ready to eat beverage cakes with ingredients offer the consumers an easy and convenient way to obtain an affordable, nutritious, and delicious meal.

A novel ready to eat cold beverage cake product is created comprising: beverage cake, having at least one beverage, and at least one binding agent.

The ready to eat cold beverage cake product comprising beverage cake is soft in texture, high in nutrition, and suitable for consumption by everybody, including animal, infant, small children, elderly people, and sick people.

The ready to eat cold beverage cake product comprising beverage cake can be served as breakfast, lunch, snack, and dinner.

The ready to eat cold beverage cake product comprises beverage cake with gelatin as at least one binding agent can be refrigerated to serve cold but not reheated, because gelatin from fish and animal congeals below 15 degrees Celsius (59 degrees Fahrenheit). The beverage cake with at least one binding agent selected from the group consisting of beaten egg, egg white, and starch component can be served at room temperature, refrigerated to serve cold, and reheated to serve hot.

At least one beverage and at least one binding agent are stirred and mixed as an admixture. The beverage cake is cooked by method selected from the group consisting of deep frying, boiling, steaming, roasting, frying, grilling, and baking.

At least one beverage is selected from group consisting of beverage, distilled water, mineral water, sports drink, tea drink, flower tea, root tea, herbal tea, mushroom tea, green tea, oolong tea, black tea, white tea, yellow tea, earl grey, darjeeling, coffee drink, soup stock, roasted cereal grain beverage, roasted nut and seed beverage, roasted bean beverage, malted drink, fruit juice, vegetable juice, fruit drink, vegetable drink, fruit skin drink, vegetable skin drink, plant drink, herb drink, soft drink, fruit punch, beer, wine, liquor, spirit, cocktail, alcoholic drink, yogurt drink, milk drink, mammal milk, plant milk, milk shake, whole milk, lowfat milk, nonfat milk, lactaid milk, chocolate milk, buttermilk, malted milk, soy milk, coconut milk, almond milk, and vanilla milk.

At least one binding agent is an edible binder and is selected from the group consisting of protein component and starch component. The protein component having ingredients selected from the group consisting of gelatin from fish and animal, beaten egg, and egg white. The starch component having ingredient selected form group consisting of vegetable gum, vegetable starch, and vegetable flour. The vegetable gum having ingredient selected from the group consisting of agar, alginic acid, sodium alginate, carrageenan, gum arabic, gum ghatti, gum tragacanth, karaya gum, guar gum, locust bean gum, beta glucan, chicle gum, dammar gum, glucomannan gum, mastic gum, psyllium seed husk, spruce gum, tara gum, gellan gum, and xanthan gum. The vegetable starch and vegetable flour having ingredient selected from the group consisting of root vegetable, cereal grain, nut, seed, bean, pectin, sago, and tapioca. The ingredient can be cooked by method selected from the group consisting of mixing with water, boiling, and steaming. The gelatin from fish and animal congeals below 15 degrees Celsius (59 degrees Fahrenheit).

At least one binding agent selected from the group consisting of beaten egg and egg white is used to create beverage egg cake. The beverage egg cake can use milk as the beverage for making egg cake. The beverage egg cake can also use other kind of beverage or a mixture of other kinds of beverage for making egg cake. The beverage egg cake can be cooked by method selected from the group consisting of deep frying, roasting, baking, steaming, boiling, grilling, and frying.

The beverage egg cake is created in form selected from the group consisting of having no outer layer; having an outer layer of casing selected from the group consisting of intestine, and cellulose; comprising at least one outer coating selected from the group consisting of bread crumbs, beer batter, egg batter, cornmeal batter, milk batter, yogurt batter, tea batter, coffee batter, malt batter, chocolate batter, honey batter, syrup batter, caramel batter, butterscotch batter, peanut butter batter, butter batter, margarine batter, cheese batter, cereal grain batter, nut batter, seed batter, bean batter, vegetable batter, plant batter, herb batter, fruit batter, wine batter, liquor batter, alcoholic batter, soft drink batter, soup batter, plant wax, animal wax, beeswax, carnauba wax, donut, roll, pie, pastry, waffle, crepe, pancake, pita, tortilla, bread, taco, biscuit, cookie, cake, jelly, royal jelly, cheese, cream cheese, cottage cheese, mozzarella, mascarpone, ricotta, butter, margarine, pudding, custard, candy, ice cream, gelato, sherbet, yogurt, chocolate, and fudge; having no interior filing; comprising at least one interior filing of various ingredient; having an even composition; having an uneven composition; having horizontal layers of varying composition; having vertical layers of varying composition; and having diagonal layers of varying composition.

The beverage cake further comprising at least one flavoring ingredient causes the beverage cake to be flavored for consumption, and selected from the group consisting of natural flavor, artificial flavor, fruit flavor, flower flavor, flower bud flavor, pollen flavor, leaf flavor, stem flavor, stem shoot flavor, bulb flavor, plant flavor, root flavor, herb flavor, tea flavor, nut flavor, seed flavor, bean flavor, cereal grain flavor, fruit skin flavor, vegetable oil flavor, vegetable fat flavor, vegetable skin flavor, and vegetable flavor, including hop, menthol, cordyceps, echinacea, elder, horehound, mallow, sage, thyme, cowslip, burnet, yarrow, marsh mallow, lady's mantle, speedwell, plantain, linden flowers, wild thyme, hyssop, eucalyptus, sea buckthorn, elder blossom, verbena, lemon balm, clove, coriander, cumin, basil, dill, rosemary, parsley, fennel, oregano, allspice, catnip, wormwood, musk, chili, horseradish, tumeric, mustard, curry, wasabi, mountain mint, peppermint, spearmint, wintergreen, cinnamon, licorice, rose, jasmine, chamomile, chrysanthemum, lavender, acacia, safflower, vanilla, alfafa, apple, orange, lemon, lime, grapefruit, grape, guava, plum, peach, pear, blackberry, blueberry, strawberry, cranberry, raspberry, cherry, mango, pineapple, pomegranate, papaya, watermelon, rockmelon, hamimelon, melon, honeydew, coconut, durian, passion fruit, banana, fig, apricot, mandarin, blackcurrant, starfruit, kiwi, pomelo, lychee, loquat, persimmon, tangerine, mangosteen, noni, sugar apple, mulberry, elderberry, gooseberry, lingonberry, juniper berry, jackfruit, dragonfruit, abiu, acai, acerola, ackee, cherry orange, moringa, ambarella, araza, arhat, atemoya, atherton raspberry, babaco, bacupari, bacuri, bael, barbadine, beach plum, batuan, bearberry, bignay, bilberry, bilimbi, biriba, black apple, black cherry, black raspberry, black mulberry, black sapote, blood orange, bolwarra, boquila, blue tongue, breadfruit, broad leaf bramble, buddha's hand, buffaloberry, brush cherry, burdekin plum, bush tomato, button mangosteen, caimito, calabash tree, calamondin, calamansi, camucamu, canary melon, canistel, cape gooseberry, capuli cherry, carambola, cardon, carob, cashew apple, cassabanana, cattley guava, cawesh, cedar bay cherry, ceriman, cereus peruvianus, charichuelo, chayote, che, chempedak, chenet, cherimoya, cherry ballart, chokeberry, chokecherry, chupa chupa, citron, clementine, cloudberry, cluster fig, cocky apple, coco plum, cocona, cocoplum, coffee cherry, common apple berry, conkerberry, cornelian cherry, crowberry, cupuacu, currant, curry leaf apple, custard apple, elephant apple, key apple, damson plum, date, date plum, davidson's plum, dead man's finger, desert banana, desert fig, desert lime, dodder laurel, doubah, double coconut, duku, eastern may hawthorn, emu apple, emu berry, false mastic, feijoa, fibrous satinash, finger lime, florida strangler fig, gac, galia melon, gambooge, genip, giant granadilla, otaheite gooseberry, goumi, governor's plum, montessa granadilla, yellow granadilla, greengage, grumichama, guanabana, guarana, cattley guava, pineapple guava, purple guava, strawberry guava, guavaberry, hackberry, hairless rambutan, hardy kiwi, hawthorn, hog plum, honeysuckle, horned melon, huckleberry, huito, ice cream bean, llama, lllawarra plum, imbe, jabuticaba, jambul, jatoba, jelly palm, jocote, jujube, kabosu, kaffir lime, kahikatea, kakadu lime, kakadu plum, kapok, karkalla, karonda, kei apple, kepel fruit, keule, key lime, kitembilla, korlan, kumquat, kutjera, kwai muk, lady apple, lakoocha, langsat, lanzone, lapsi, lardizabala, lemon aspen, leucaena, lillypilly, little gooseberry tree, limequat, loganberry, longan, lucuma, ma praang, mabolo, madrono, malabar plum, mamey sapote, mamoncillo, mangaba, manoao, maqui, marang, manila, mayapple, malay apple, mammee apple, maypop, medlar, melinjo, melon pear, midyim, miracle fruit, mock strawberry, purple mombin, red mombin, yellow mombin, monstera, mora de castilla, morinda, morinda citrifolia, mortiflo, mountain pepper, mountain soursop, wild plum, mundu, muntries, muscadine, muskmelon, naartjie, nageia, nance, nannyberry, naranjilla, neem, nere, nonda plum, nungu, oil palm, oregan grape, oroblanco, otaheite gooseberry, palmyra palm, pawpaw, peachpalm, peanut butter fruit, pepino, pequi, peumo, phalsa, pigeon pea, pigeon plum, pigface, pindo palm, pineapple guava, pitomba, pois doux, pommecythere, pommerac, prickly pear, prune, pulasan, pummelo, pupunha, purple apple berry, purple granadilla, quandong, queensland ebony, quince, raisin tree, rambutan, red granadilla, red mulberry, rhubarb, riberry, rimu, rollinia, rose apple, rose hip, rose leaf bramble, rose myrtle, rowan, safou, sageretia, saguaro, salak, salal, salmonberry, sandpaper fig, santol, mamey, sapodilla, fibrous satinash, saskatoonberry, saw palmetto, sea buckthorn, serviceberry, shipova, small leaf tamarind, snow berry, soncoya, servicetree, soursop, southern crabapple, spanish lime, spanish tamarind, spiny monkey orange, star apple, carambola, strawberry guava, ugni, strawberry pear, surinam cherry, sweet apple berry, sweet ganadilla, sweet lemon, sweet orange, sweet pepper, sweetsop, sycamore fig, tamarillo, tamarind, monkey tamarind, velvet tamarind, tanjong, texas persimmon, thimbleberry, totara, toyon, wampee, water apple, wax apple, wax jambu, acerola, white aspen, white mulberry, white sapote, wild grape, wild orange, wild peach, wild plum, wild mangosteen, wineberry, wolfberry, wongi, wood apple, yangmei, yellow plum, yew, youngberry, zig zag vine, ahipa, arracacha, bamboo shoot, beetroot, burdock, broadleaf arrowhead, camas, canna, carrot, cassava, celeriac, chinese artichoke, daikon, earthnut pea, elephant foot yam, ensete, galangal, ginger, ginseng, hamberg parsley, horseradish, lotus root, jerusalem artichoke, jicama, mashua, parsley root, parsnip, pignut, potato, prairie turnip, radish, rutabaga, salsify, scorzonera, skirret, swede, sweet potato, taro, ti, tigernut, tumeric, turnip, ulluco, wasabi, water caltrop, water chestnut, yalcon, yam, avocado, bell pepper, bitter melon, bitter gourd, breadfruit, chayote, cucumber, ivy gourd, eggplant, aubergine, brinjal, luffa, nopal, okra, pepper, pumpkin, squash, sweet corn, sweet pepper, tinda, tomatillo, tomato, west indian gherkin, winter melon, zucchini, courgette, artichoke, broccoli, caper, cauliflower, courgette flower, globe artichoke, squash blossom, asparagus, prussian asparagus, bamboo shoot, celery, leek, wild leek, lemongrass, rhubarb, cardoon, kurrat, galangal, amaranth, arugula, beet green, bok choy, borage, broccoli rabe, brussel sprout, cabbage, catsear, celery, celtuce, chard, chaya, chickweed, chicory, chinese mallow, chive, choi sum, chrysanthemum, collard green, common purslane, corn salad, cress, dandelion, endive, fat hen, fiddlehead, fluted pumpkin, garden rocket, golden samphire, good king henry, greater plantain, kai lan, kale, komatsuna, kuka, lagos bologi, lamb's lettuce, lamb's quarters, land cress, leek, lettuce, lizard's tail, malabar spinach, melokhia, miner's lettuce, mizuna green, mustard green, napa cabbage, new zealand spinach, orache, pak choy, paracress, pea, poke, radicchio, samphire, sculpit, stridolo, sea beet, sea kale, sierra leoni bologi, soko, sorrel, spinach, summer purslane, swiss chard, tatsoi, turnip green, watercress, water spinach, wheatgrass, winter purslane, yarrow, yao choy, cardoon, chive, florence fennel, elephant garlic, garlic, garlic chive, kohlrabi, onion, pearl onion, potato onion, spring onion, tree onion, welsh onion, quamash, scallion, shallot, rice, barley, oat, rye, corn, wheat, buckwheat, millet, sorghum, spelt, quinoa, chicory, molasses, beet root, fonio, palmer's grass, teff, triticale, cattail, chia, flax, grain amaranth, canihua, pitseed goosefoot, wattleseed, almond, cashew, chestnut, coconut, hazelnut, macadamia, peanut, pecan, pine nut, pistachio, walnut, acorn, brazil nut, gingko nut, shea nut, beechnut, candlenut, kurrajong, mongongo, palm nut, karuka, red bopple nut, hickory, jacknut, pepea nut, yellow walnut, cycad, munya nut, monkey puzzle nut, paradise nut, colocynth, filbert, mamoncillo, maya nut, ogbono nut, water caltrop, juniper, podocarp, cempedak, coffee, egusi, fox nut, jackfruit, malabar gourd, nutmeg, breadnut, betel nut, cola nut, pili nut, olive, drumstick, okra, pumpkin seed, sesame seed, sunflower seed, lotus seed, hemp seed, green bean, red bean, azuki bean, black bean, back eyed bean, white bean, yellow bean, common bean, broad bean, pinto bean, soybean, lima bean, kidney bean, pea bean, potato bean, dolichos bean, moth bean, mung bean, ricebean, runner bean, tepary bean, urad bean, velvet bean, winged bean, yardlong bean, chickpea, pigeon pea, snap pea, snow pea, lentil, lupin, tarwi, carob, guar, horse gram, cocoa bean, coffee bean, bambara groundnut, cowpea, hyacinth bean, lablab, moringa oleifera, sterculia, yam bean, alfalfa, clover, mesquite, carob, tamarind, wisteria, aonori, arame, carola, caulerpa, dabberlocks, dulse, hijiki, kombu, laver, mozuku, nori, ogonori, sea grape, sea lettuce, sea weed, wakame, truffle, mushroom, bean sprout, acai, aloe vera, arnica, ashoka, asthma weed, astragalus, barberry, belladonna, bitter leaf, bitter orange, black cohosh, blessed thistle, cat's claw, cayenne, chaparral, chasteberry, cinchona, coffee senna, comfrey, digitalis, dong quai, ephedra, european mistletoe, evening primrose, fenugreek, feverfew, flaxseed, gingko, goldenseal, hoodia, horse chestnut, horsetail, jamaica dogwood, kava, khat, konjac, kratom, kanna, licorice root, marigold, moringa oleifera, milk thistle, neem, opium poppy, purple coneflower, passion flower, red clover, syrian rue, st john's wort, saw palmetto, tea tree oil, thunder god vine, tulasi, turmeric, umckaloabo, valerian, white willow, yerba santa, ajwain, akudjura, alexanders, alkanet, alligator pepper, angelica, anise, anise hyssop, aniseed myrtle, annatto, apple mint, artemisia, asafoetida, asarabacca, avens, avocado leaf, barberry, bay leaf, bee balm, boldo, borage, black cardamom, blue fenugreek, caraway, cardamom, cassia, cayenne pepper, celery leaf, celery seed, chervil, chicory, cicely, cilantro, clary, costmary, cubeb pepper, cudweed, culantro, curry leaf, curry plant, dill seed, epazote, fenugreek, file powder, fingerroot, galangal, galingale, ganoderma, golpar, grains of paradise, grains of selim, houttuynia cordata, huacatay, Indonesian bay leaf, jimbu, kaffir lime leaves, kala zeera, kawakawa seeds, kencur, keluak, kinh gioi, kokam seed, korarima, koseret leaves, lemon myrtle, lemon ironbark, lemon verbena, leptotes bicolor, lesser calamint, lime flower, lovage, mahlab, marjoram, mastic, mountain horopito, musk mallow, nasturtium, nigella, njangsa, neem, olida, orris root, pandan flower, pandan leaf, paprika, paracress, peppermint gum leaf, perilla, pandanus amaryllifolius, quassia, ramsons, rice paddy herb, rue, saffron, salad burnet, salep, sassafras, savory, silphium, shiso, sorrel, spikenard, star anise, sumac, sweet woodruff, tarragon, voatsiperifery, water-pepper, wattleseed, wild betel, willow herb, wood avens, and woodruff. At least one flavoring ingredient is used for beverage cake in manner selected from the group consisting of outer coating, interior filing, and mixed in. At least one flavoring ingredient is in form selected from the group consisting of skin, skinned, unskinned, unshelled, cut, uncut, extract, juice, sauce, liquid, oil, gel, jelly, wax, cream, paste, powder, thin layer, and small pieces. At least one flavoring ingredient can be uncooked, and cooked by method selected from the group consisting of deep frying, sun drying, roasting, baking, steaming, boiling, grilling, and frying.

The beverage cake further comprising at least one fruit flavor to make fruity flavored cake selected from the group consisting of apple, orange, lemon, lime, grapefruit, grape, guava, plum, peach, pear, blackberry, blueberry, strawberry, cranberry, raspberry, cherry, mango, pineapple, pomegranate, papaya, watermelon, rockmelon, hamimelon, melon, honeydew, coconut, durian, passion fruit, banana, fig, apricot, mandarin, blackcurrant, starfruit, kiwi, pomelo, lychee, loquat, persimmon, tangerine, mangosteen, noni, sugar apple, mulberry, elderberry, gooseberry, lingonberry, juniper berry, jackfruit, dragonfruit, abiu, acai, acerola, ackee, cherry orange, moringa, ambarella, araza, arhat, atemoya, atherton raspberry, babaco, bacupari, bacuri, bael, barbadine, beach plum, batuan, bearberry, bignay, bilberry, bilimbi, biriba, black apple, black cherry, black raspberry, black mulberry, black sapote, blood orange, bolwarra, boquila, blue tongue, breadfruit, broad leaf bramble, buddha's hand, buffaloberry, brush cherry, burdekin plum, bush tomato, button mangosteen, caimito, calabash tree, calamondin, calamansi, camucamu, canary melon, canistel, cape gooseberry, capuli cherry, carambola, cardon, carob, cashew apple, cassabanana, cattley guava, cawesh, cedar bay cherry, ceriman, cereus peruvianus, charichuelo, chayote, che, chempedak, chenet, cherimoya, cherry ballart, chokeberry, chokecherry, chupa chupa, citron, clementine, cloudberry, cluster fig, cocky apple, coco plum, cocona, cocoplum, coffee cherry, common apple berry, conkerberry, cornelian cherry, crowberry, cupuacu, currant, curry leaf apple, custard apple, elephant apple, key apple, damson plum, date, date plum, davidson's plum, dead man's finger, desert banana, desert fig, desert lime, dodder laurel, doubah, double coconut, duku, eastern may hawthorn, emu apple, emu berry, false mastic, feijoa, fibrous satinash, finger lime, florida strangler fig, gac, galia melon, gambooge, genip, giant granadilla, otaheite gooseberry, goumi, governor's plum, montessa granadilla, yellow granadilla, greengage, grumichama, guanabana, guarana, cattley guava, pineapple guava, purple guava, strawberry guava, guavaberry, hackberry, hairless rambutan, hardy kiwi, hawthorn, hog plum, honeysuckle, horned melon, huckleberry, huito, ice cream bean, llama, lllawarra plum, imbe, jabuticaba, jambul, jatoba, jelly palm, jocote, jujube, kabosu, kaffir lime, kahikatea, kakadu lime, kakadu plum, kapok, karkalla, karonda, kei apple, kepel fruit, keule, key lime, kitembilla, korlan, kumquat, kutjera, kwai muk, lady apple, lakoocha, langsat, lanzone, lapsi, lardizabala, lemon aspen, leucaena, lillypilly, little gooseberry tree, limequat, loganberry, longan, lucuma, ma praang, mabolo, madrono, malabar plum, mamey sapote, mamoncillo, mangaba, manoao, maqui, marang, manila, mayapple, malay apple, mammee apple, maypop, medlar, melinjo, melon pear, midyim, miracle fruit, mock strawberry, purple mombin, red mombin, yellow mombin, monstera, mora de castilla, morinda, morinda citrifolia, mortiflo, mountain pepper, mountain soursop, wild plum, mundu, muntries, muscadine, muskmelon, naartjie, nageia, nance, nannyberry, naranjilla, neem, nere, nonda plum, nungu, oil palm, oregan grape, oroblanco, otaheite gooseberry, palmyra palm, pawpaw, peachpalm, peanut butter fruit, pepino, pequi, peumo, phalsa, pigeon pea, pigeon plum, pigface, pindo palm, pineapple guava, pitomba, pois doux, pommecythere, pommerac, prickly pear, prune, pulasan, pummelo, pupunha, purple apple berry, purple granadilla, quandong, queensland ebony, quince, raisin tree, rambutan, red granadilla, red mulberry, rhubarb, riberry, rimu, rollinia, rose apple, rose hip, rose leaf bramble, rose myrtle, rowan, safou, sageretia, saguaro, salak, salal, salmonberry, sandpaper fig, santol, mamey, sapodilla, fibrous satinash, saskatoonberry, saw palmetto, sea buckthorn, serviceberry, shipova, small leaf tamarind, snow berry, soncoya, servicetree, soursop, southern crabapple, spanish lime, spanish tamarind, spiny monkey orange, star apple, carambola, strawberry guava, ugni, strawberry pear, surinam cherry, sweet apple berry, sweet ganadilla, sweet lemon, sweet orange, sweet pepper, sweetsop, sycamore fig, tamarillo, tamarind, monkey tamarind, velvet tamarind, tanjong, texas persimmon, thimbleberry, totara, toyon, wampee, water apple, wax apple, wax jambu, acerola, white aspen, white mulberry, white sapote, wild grape, wild orange, wild peach, wild plum, wild mangosteen, wineberry, wolfberry, wongi, wood apple, yangmei, yellow plum, yew, youngberry, and zig zag vine. At least one fruit flavor is used for beverage cake in manner selected from the group consisting of outer coating, interior filing, and mixed in. At least one fruit flavor is in form selected from the group consisting of skin, skinned, unskinned, unshelled, cut, uncut, extract, juice, sauce, liquid, oil, gel, jelly, wax, cream, paste, powder, thin layer, and small pieces. At least one fruit flavor can be uncooked, and cooked by method selected from the group consisting of deep frying, sun drying, roasting, baking, steaming, boiling, grilling, and frying.

The beverage cake further comprises at least one other flavoring ingredient selected from the group consisting of natural flavor, artificial flavor, artificial sweetener, salt, vinegar, sugar, syrup, honey, fudge, chocolate, royal jelly, jelly, pudding, custard, ice cream, sherbet, gelato, cake, biscuit, cookie, crepe, pancake, donut, roll, pie, pastry, waffle, pita, tortilla, bread, taco, candy, marshmallow, caramel, butterscotch, raisin, apple sauce, vegetable shortening, vegetable oil, vegetable fat, fish oil, fish liver oil, plant wax, animal wax, beeswax, carnauba wax, cream, sour cream, yogurt, butter, margarine, peanut butter, cheese, cream cheese, cottage cheese, mozzarella, mascarpone, ricotta, egg, glycerol, suet, lard, and malt. At least one other flavoring ingredient is used for beverage cake in manner selected from the group consisting of outer coating, interior filing, and mixed in. At least one other flavoring ingredient is in form selected from the group consisting of extract, juice, sauce, liquid, oil, gel, jelly, wax, cream, paste, powder, thin layer, and small pieces.

The beverage cake further comprising at least one nutritional ingredient which is essential nutrient selected from the group consisting of vitamin, dietary mineral, amino acid, and fatty acid. The vitamin includes vitamin A, vitamin B1, vitamin B2, vitamin B3, vitamin B5, vitamin B6, vitamin B7, vitamin B9, vitamin B12, vitamin C, vitamin D, vitamin E, and vitamin K. The dietary mineral includes calcium, phosphorus, potassium, sulfur, sodium, chlorine, magnesium, iron, cobalt, copper, zinc, molybdenum, iodine, selenium, manganese, nickle, chromium, fluorine, boron, and strontium. The amino acid includes histidine, isoleucine, leucine, lysine, methionine, cysteine, phenylalanine, tyrosine, threonine, tryptophan, and valine; said fatty acid includes alpha-linoleic acid, and linoleic acid. At least one nutritional ingredient is used for beverage cake in manner selected from the group consisting of outer coating, interior filing, and mixed in. At least one nutritional ingredient is in form selected from the group consisting of extract, juice, sauce, liquid, oil, gel, jelly, wax, cream, paste, powder, thin layer, and small pieces.

The beverage cake further comprising at least one health ingredient which includes medication and other health ingredient. The medication is mixed into the beverage cake for ease of consumption. The other health ingredient is selected from the group consisting of electrolyte, lactase, yeast, propolis, and quinine. At least one health ingredient is in form selected from the group consisting of extract, juice, sauce, liquid, oil, gel, jelly, wax, cream, paste, powder, thin layer, and small pieces.

The beverage cake further comprising at least one food additive selected from the group consisting of acid, acidity regulator, alcohol, anticaking agent, antifoaming agent, antioxidant, bulking agent, coagulation agent, food coloring, color retention agent, emulsifier, flavor enhancer, flour treatment agent, gelling agent, glazing agent, humectant, leavening agent, tracer gas, preservative, stabilizer, sweetener, and thickener.

The beverage cake further comprising at least one topping selected from the group consisting of chocolate, caramel, butterscotch, peanut butter, butter, margarine, vegetable shortening, vegetable oil, vegetable fat, fish oil, fish liver oil, plant wax, animal wax, beeswax, carnauba wax, sugar, syrup, honey, royal jelly, apple sauce, raisin, fruit, vegetable, fruit skin, vegetable skin, herb, cereal grain, nut, seed, bean, cheese, cream cheese, cottage cheese, mozzarella, mascarpone, ricotta, jelly, pudding, custard, ice cream, yogurt, sherbet, gelato, cake, biscuit, cookie, crepe, pancake, donut, roll, pie, pastry, waffle, pita, tortilla, bread, taco, candy, marshmallow, sour cream, cream, malt, egg, meat, and seafood. The topping is in form selected from the group consisting of extract, juice, sauce, liquid, oil, gel, jelly, wax, cream, paste, powder, thin layer, and small pieces. The topping can be uncooked, and cooked by method selected from the group consisting of deep frying, sun drying, roasting, baking, steaming, boiling, grilling, and frying.

The beverage cake further comprising at least one salt source having a combination of ingredients selected from the group consisting of seafood, meat, and cheese, including seafood and meat, seafood and cheese, meat and cheese, seafood and meat and cheese. The seafood is selected from the group consisting of squid, cuttle fish, shrimp, crab, crayfish, octopus, clam, oyster, scallop, lobster, abalone, conch, jellyfish, prawn, krill, mussel, sea cucumber, sea urchin, eel, snail, shrimp roe, crab roe, lobster roe, fish roe, and fish. The meat is selected from the group consisting of chicken, duck, goose, turkey, quail, rabbit, sheep, goat, cow, pig, deer, bison, buffalo, crocodile, frog, turtle, snail, worm, bacon, sausage, animal tissue, animal organ, animal tongue, animal tail, animal feet, animal liver, animal stomach, animal intestine, animal brain, animal heart, animal skin, animal tendon, animal fat, animal bone marrow, and animal blood. At least one salt source is used for beverage cake in manner selected from the group consisting of outer coating, interior filing, and mixed in. At least one salt source is selected from the group consisting of cut, uncut, slice into thin layer, cut into small pieces, and grind into small particles. At least one salt source can be cooked by method selected from the group consisting of deep frying, roasting, baking, steaming, boiling, grilling, and frying.

At least one salt source having a combination of ingredients is grinded to create beverage plus salt source cake. The beverage plus salt source cake is prepared with a small amount of at least one liquid ingredient. The beverage plus salt source cake is having at least one binding agent selected from the group consisting of beaten egg, egg white, and starch component. The beverage plus salt source cake is cooked by method selected from the group consisting of deep frying, roasting, baking, boiling, steaming, grilling, and frying.

The beverage plus salt source cake is created in form selected from the group consisting of having no outer layer; having an outer layer of casing selected from the group consisting of intestine, and cellulose; comprising at least one outer coating selected from the group consisting of bread crumbs, beer batter, egg batter, cornmeal batter, milk batter, yogurt batter, tea batter, coffee batter, malt batter, chocolate batter, honey batter, syrup batter, caramel batter, butterscotch batter, peanut butter batter, butter batter, margarine batter, cheese batter, cereal grain batter, nut batter, seed batter, bean batter, vegetable batter, plant batter, herb batter, fruit batter, wine batter, liquor batter, alcoholic batter, soft drink batter, soup batter, plant wax, animal wax, beeswax, carnauba wax, donut, roll, pie, pastry, waffle, crepe, pancake, pita, tortilla, bread, taco, biscuit, cookie, cake, jelly, royal jelly, cheese, cream cheese, cottage cheese, mozzarella, mascarpone, ricotta, butter, margarine, pudding, custard, candy, ice cream, gelato, sherbet, yogurt, chocolate, and fudge; having no interior filing; comprising at least one interior filing of various ingredient; having an even composition; having an uneven composition; having horizontal layers of varying composition; having vertical layers of varying composition; and having diagonal layers of varying composition.

The beverage cake further comprising at least one seafood flavor selected from the group consisting of squid, cuttle fish, shrimp, crab, crayfish, octopus, clam, oyster, scallop, lobster, abalone, conch, jellyfish, prawn, hill, mussel, sea cucumber, sea urchin, eel, snail, shrimp roe, crab roe, lobster roe, fish roe, and fish. At least one seafood flavor is used for beverage cake in manner selected from the group consisting of outer coating, interior filing, and mixed in. At least one seafood flavor is selected from the group consisting of cut, uncut, slice into thin layer, cut into small pieces, and grind into small particles. At least one seafood flavor can be cooked by method selected from the group consisting of deep frying, roasting, baking, steaming, boiling, grilling, and frying.

At least one seafood is grinded and used as ingredient to create beverage seafood cake. The beverage seafood cake is prepared with a small amount of at least one liquid ingredient. The beverage seafood cake is having at least one binding agent selected from the group consisting of beaten egg, egg white, and starch component. The beverage seafood cake is cooked by method selected from the group consisting of roasting, baking, boiling, steaming, grilling, and frying.

The beverage seafood cake is created in form selected from the group consisting of having no outer layer; having an outer layer of casing selected from the group consisting of intestine, and cellulose; comprising at least one outer coating selected from the group consisting of bread crumbs, beer batter, egg batter, cornmeal batter, milk batter, yogurt batter, tea batter, coffee batter, malt batter, chocolate batter, honey batter, syrup batter, caramel batter, butterscotch batter, peanut butter batter, butter batter, margarine batter, cheese batter, cereal grain batter, nut batter, seed batter, bean batter, vegetable batter, plant batter, herb batter, fruit batter, wine batter, liquor batter, alcoholic batter, soft drink batter, soup batter, plant wax, animal wax, beeswax, carnauba wax, donut, roll, pie, pastry, waffle, crepe, pancake, pita, tortilla, bread, taco, biscuit, cookie, cake, jelly, royal jelly, cheese, cream cheese, cottage cheese, mozzarella, mascarpone, ricotta, butter, margarine, pudding, custard, candy, ice cream, gelato, sherbet, yogurt, chocolate, and fudge; having no interior filing; comprising at least one interior filing of various ingredient; having an even composition; having an uneven composition; having horizontal layers of varying composition; having vertical layers of varying composition; and having diagonal layers of varying composition.

The beverage cake further comprising at least one meat flavor selected from the group consisting of chicken, duck, goose, turkey, quail, rabbit, sheep, goat, cow, pig, deer, bison, buffalo, crocodile, frog, turtle, snail, worm, bacon, sausage, animal tissue, animal organ, animal tongue, animal tail, animal feet, animal liver, animal stomach, animal intestine, animal brain, animal heart, animal skin, animal tendon, animal fat, animal bone marrow, and animal blood. At least one meat flavor is used for beverage cake in manner selected from the group consisting of outer coating, interior filing, and mixed in. At least one meat flavor is selected from the group consisting of cut, uncut, slice into thin layer, cut into small pieces, and grind into small particles. At least one meat flavor can be cooked by method selected from the group consisting of deep frying, roasting, baking, steaming, boiling, grilling, and frying.

At least one meat is grinded and used as ingredient to create beverage meat cake. The beverage meat cake is prepared with a small amount of said at least one liquid ingredient. The beverage meat cake is having at least one binding agent selected from the group consisting of egg, egg white, and starch component. The beverage meat cake is cooked by method selected from the group consisting of deep frying, roasting, baking, boiling, steaming, grilling, and frying.

The beverage meat cake is created in form selected from the group consisting of having no outer layer; having an outer layer of casing selected from the group consisting of intestine, and cellulose; comprising at least one outer coating selected from the group consisting of bread crumbs, beer batter, egg batter, cornmeal batter, milk batter, yogurt batter, tea batter, coffee batter, malt batter, chocolate batter, honey batter, syrup batter, caramel batter, butterscotch batter, peanut butter batter, butter batter, margarine batter, cheese batter, cereal grain batter, nut batter, seed batter, bean batter, vegetable batter, plant batter, herb batter, fruit batter, wine batter, liquor batter, alcoholic batter, soft drink batter, soup batter, plant wax, animal wax, beeswax, carnauba wax, donut, roll, pie, pastry, waffle, crepe, pancake, pita, tortilla, bread, taco, biscuit, cookie, cake, jelly, royal jelly, cheese, cream cheese, cottage cheese, mozzarella, mascarpone, ricotta, butter, margarine, pudding, custard, candy, ice cream, gelato, sherbet, yogurt, chocolate, and fudge; having no interior filing; comprising at least one interior filing of various ingredient; having an even composition; having an uneven composition; having horizontal layers of varying composition; having vertical layers of varying composition; and having diagonal layers of varying composition.

The beverage cake further comprising at least one vegetable flavor selected from the group consisting of root vegetable, fruit vegetable, flower bud, flower, stem shoot, stem, leaf, bulb, cereal grain, nut, seed, bean, sea vegetable, and other types of vegetable, including ahipa, arracacha, bamboo shoot, beetroot, burdock, broadleaf arrowhead, camas, canna, carrot, cassava, celeriac, chinese artichoke, daikon, earthnut pea, elephant foot yam, ensete, galangal, ginger, ginseng, hamberg parsley, horseradish, lotus root, jerusalem artichoke, jicama, mashua, parsley root, parsnip, pignut, potato, prairie turnip, radish, rutabaga, salsify, scorzonera, skirret, swede, sweet potato, taro, ti, tigernut, tumeric, turnip, ulluco, wasabi, water caltrop, water chestnut, yalcon, yam, avocado, bell pepper, bitter melon, bitter gourd, breadfruit, chayote, cucumber, ivy gourd, eggplant, aubergine, brinjal, luffa, nopal, okra, pepper, pumpkin, squash, sweet corn, sweet pepper, tinda, tomatillo, tomato, west indian gherkin, winter melon, zucchini, courgette, artichoke, broccoli, caper, cauliflower, courgette flower, globe artichoke, squash blossom, asparagus, prussian asparagus, bamboo shoot, celery, leek, wild leek, lemongrass, rhubarb, cardoon, kurrat, galangal, amaranth, arugula, beet green, bok choy, borage, broccoli rabe, brussel sprout, cabbage, catsear, celery, celtuce, chard, chaya, chickweed, chicory, chinese mallow, chive, choi sum, chrysanthemum, collard green, common purslane, corn salad, cress, dandelion, endive, fat hen, fiddlehead, fluted pumpkin, garden rocket, golden samphire, good king henry, greater plantain, kai lan, kale, komatsuna, kuka, lagos bologi, lamb's lettuce, lamb's quarters, land cress, leek, lettuce, lizard's tail, malabar spinach, melokhia, miner's lettuce, mizuna green, mustard green, napa cabbage, new zealand spinach, orache, pak choy, paracress, pea, poke, radicchio, samphire, sculpit, stridolo, sea beet, sea kale, sierra leoni bologi, soko, sorrel, spinach, summer purslane, swiss chard, tatsoi, turnip green, watercress, water spinach, wheatgrass, winter purslane, yarrow, yao choy, cardoon, chive, florence fennel, elephant garlic, garlic, garlic chive, kohlrabi, onion, pearl onion, potato onion, spring onion, tree onion, welsh onion, quamash, scallion, shallot, rice, barley, oat, rye, corn, wheat, buckwheat, millet, sorghum, spelt, quinoa, chicory, molasses, beet root, fonio, palmer's grass, teff, triticale, cattail, chia, flax, grain amaranth, canihua, pitseed goosefoot, wattleseed, almond, cashew, chestnut, coconut, hazelnut, macadamia, peanut, pecan, pine nut, pistachio, walnut, acorn, brazil nut, gingko nut, shea nut, beechnut, candlenut, kurrajong, mongongo, palm nut, karuka, red bopple nut, hickory, jacknut, pepea nut, yellow walnut, cycad, munya nut, monkey puzzle nut, paradise nut, colocynth, filbert, mamoncillo, maya nut, ogbono nut, water caltrop, juniper, podocarp, cempedak, coffee, egusi, fox nut, jackfruit, malabar gourd, nutmeg, breadnut, betel nut, cola nut, pili nut, olive, drumstick, okra, pumpkin seed, sesame seed, sunflower seed, lotus seed, hemp seed, green bean, red bean, azuki bean, black bean, back eyed bean, white bean, yellow bean, common bean, broad bean, pinto bean, soybean, lima bean, kidney bean, pea bean, potato bean, dolichos bean, moth bean, mung bean, ricebean, runner bean, tepary bean, urad bean, velvet bean, winged bean, yardlong bean, chickpea, pigeon pea, snap pea, snow pea, lentil, lupin, tarwi, carob, guar, horse gram, cocoa bean, coffee bean, bambara groundnut, cowpea, hyacinth bean, lablab, moringa oleifera, sterculia, yam bean, alfalfa, clover, mesquite, carob, tamarind, wisteria, aonori, arame, carola, caulerpa, dabberlocks, dulse, hijiki, kombu, laver, mozuku, nori, ogonori, sea grape, sea lettuce, sea weed, wakame, truffle, mushroom, and bean sprout. At least one vegetable flavor is used for beverage cake in manner selected from the group consisting of outer coating, interior filing, and mixed in. At least one vegetable flavor is in form selected from the group consisting of skin, skinned, unskinned, unshelled, cut, uncut, extract, juice, sauce, liquid, oil, gel, jelly, wax, cream, paste, powder, thin layer, and small pieces. At least one vegetable flavor can be uncooked, and cooked by method selected from the group consisting of deep frying, sun drying, roasting, baking, steaming, boiling, grilling, and frying.

The beverage cake is created in form selected from the group consisting of having no outer layer; having an outer layer of casing selected from the group consisting of intestine, and cellulose; comprising at least one outer coating selected from the group consisting of bread crumbs, beer batter, egg batter, cornmeal batter, milk batter, yogurt batter, tea batter, coffee batter, malt batter, chocolate batter, honey batter, syrup batter, caramel batter, butterscotch batter, peanut butter batter, butter batter, margarine batter, cheese batter, cereal grain batter, nut batter, seed batter, bean batter, vegetable batter, plant batter, herb batter, fruit batter, wine batter, liquor batter, alcoholic batter, soft drink batter, soup batter, plant wax, animal wax, beeswax, carnauba wax, donut, roll, pie, pastry, waffle, crepe, pancake, pita, tortilla, bread, taco, biscuit, cookie, cake, jelly, royal jelly, cheese, cream cheese, cottage cheese, mozzarella, mascarpone, ricotta, butter, margarine, pudding, custard, candy, ice cream, gelato, sherbet, yogurt, chocolate, and fudge; having no interior filing; comprising at least one interior filing of various ingredient; having an even composition; having an uneven composition; having horizontal layers of varying composition; having vertical layers of varying composition; and having diagonal layers of varying composition.

The beverage cake having a variety of shapes and sizes is selected from the group consisting of sphere, disc, tube, cube, cylinder, cone, triangle, hexagon, octagon, rectangle, and oval. The beverage cake having a variety of shapes and sizes selected from the group consisting of cubical shape with each side of about 1 to 10 inches, spherical shape with diameter of about 1 to 100 millimeters, disc shape with diameter of about 3 to 5 inches and thickness of about ½ to 2 inches, and tube shape with diameter of about ½ to 2 inches and length of about 1 to 10 inches.

The ready to eat cold beverage cake product with spherical shape having diameter of about 1 to 10 millimeters is created as beverage bubble. The beverage bubble is cooked by method selected from the group consisting of deep frying, roasting, baking, steaming, boiling, grilling, and frying.

The ready to eat cold beverage cake product with spherical shape having diameter of about 10 to 40 millimeters is created as beverage ball. The beverage ball is cooked by method selected from the group consisting of deep frying, roasting, baking, steaming, boiling, grilling, and frying.

The beverage ball is created in form selected from the group consisting of spherical shape without an outer layer; spherical shape with an outer layer of casing selected from the group consisting of intestine, and cellulose; spherical shape comprising at least one outer coating selected from the group consisting of bread crumbs, beer batter, egg batter, cornmeal batter, milk batter, yogurt batter, tea batter, coffee batter, malt batter, chocolate batter, honey batter, syrup batter, caramel batter, butterscotch batter, peanut butter batter, butter batter, margarine batter, cheese batter, cereal grain batter, nut batter, seed batter, bean batter, vegetable batter, plant batter, herb batter, fruit batter, wine batter, liquor batter, alcoholic batter, soft drink batter, soup batter, plant wax, animal wax, beeswax, carnauba wax, donut, roll, pie, pastry, waffle, crepe, pancake, pita, tortilla, bread, taco, biscuit, cookie, cake, jelly, royal jelly, cheese, cream cheese, cottage cheese, mozzarella, mascarpone, ricotta, butter, margarine, pudding, custard, candy, ice cream, gelato, sherbet, yogurt, chocolate, and fudge; having no interior filing; comprising at least one interior filing of various ingredient; having an even composition; having an uneven composition; having horizontal layers of varying composition; having vertical layers of varying composition; and having diagonal layers of varying composition.

The spherical shape and variety of shapes and sizes are mixable in drink, food, and condiment; and are mixable in ingredient selected from the group consisting of yogurt, ice cream, cake, rice, mashed potato, pasta, noodle, salad dressing, sauce, spread, beverage, distilled water, mineral water, sports drink, tea drink, flower tea, root tea, herbal tea, mushroom tea, green tea, oolong tea, black tea, white tea, yellow tea, earl grey, darjeeling, coffee drink, soup stock, roasted cereal grain beverage, roasted nut and seed beverage, roasted bean beverage, malted drink, fruit juice, vegetable juice, fruit drink, vegetable drink, fruit skin drink, vegetable skin drink, plant drink, herb drink, soft drink, fruit punch, beer, wine, liquor, spirit, cocktail, alcoholic drink, yogurt drink, milk drink, mammal milk, plant milk, milk shake, whole milk, lowfat milk, nonfat milk, lactaid milk, chocolate milk, buttermilk, malted milk, soy milk, coconut milk, almond milk, and vanilla milk.

The ready to eat cold beverage cake product with disc shape having diameter of about 1½ to 5 inches and thickness of about ⅓ to 2 inches is created as beverage patty. The beverage patty is used to make burger. The beverage patty is cooked by method selected from the group consisting of deep frying, roasting, baking, steaming, boiling, grilling, and frying.

The beverage patty is created in form selected from the group consisting of disc shape without an outer layer; disc shape with an outer layer of casing selected from the group consisting of intestine, and cellulose; disc shape comprising at least one outer coating selected from the group consisting of bread crumbs, beer batter, egg batter, cornmeal batter, milk batter, yogurt batter, tea batter, coffee batter, malt batter, chocolate batter, honey batter, syrup batter, caramel batter, butterscotch batter, peanut butter batter, butter batter, margarine batter, cheese batter, cereal grain batter, nut batter, seed batter, bean batter, vegetable batter, plant batter, herb batter, fruit batter, wine batter, liquor batter, alcoholic batter, soft drink batter, soup batter, plant wax, animal wax, beeswax, carnauba wax, donut, roll, pie, pastry, waffle, crepe, pancake, pita, tortilla, bread, taco, biscuit, cookie, cake, jelly, royal jelly, cheese, cream cheese, cottage cheese, mozzarella, mascarpone, ricotta, butter, margarine, pudding, custard, candy, ice cream, gelato, sherbet, yogurt, chocolate, and fudge; having no interior filing; comprising at least one interior filing of various ingredient; having an even composition; having an uneven composition; having horizontal layers of varying composition; having vertical layers of varying composition; and having diagonal layers of varying composition.

The ready to eat cold beverage cake product with tube shape having diameter of about ½ to 2 inches and length of about 1 to 10 inches is created as beverage sausage. The beverage sausage is used to make hot dog. The beverage sausage is cooked by method selected from the group consisting of deep frying, roasting, baking, steaming, boiling, grilling, and frying.

The beverage sausage is created in form selected from the group consisting of tube shape without an outer layer; tube shape with an outer layer of casing selected from group consisting of intestine, and cellulose; tube shape comprising at least one outer coating selected from group consisting of bread crumbs, beer batter, egg batter, cornmeal batter, milk batter, yogurt batter, tea batter, coffee batter, malt batter, chocolate batter, honey batter, syrup batter, caramel batter, butterscotch batter, peanut butter batter, butter batter, margarine batter, cheese batter, cereal grain batter, nut batter, seed batter, bean batter, vegetable batter, plant batter, herb batter, fruit batter, wine batter, liquor batter, alcoholic batter, soft drink batter, soup batter, plant wax, animal wax, beeswax, carnauba wax, donut, roll, pie, pastry, waffle, crepe, pancake, pita, tortilla, bread, taco, biscuit, cookie, cake, jelly, royal jelly, cheese, cream cheese, cottage cheese, mozzarella, mascarpone, ricotta, butter, margarine, pudding, custard, candy, ice cream, gelato, sherbet, yogurt, chocolate, and fudge; having no interior filing; comprising at least one interior filing of various ingredient; having an even composition; having an uneven composition; having horizontal layers of varying composition; having vertical layers of varying composition; and having diagonal layers of varying composition.

The ready to eat cold beverage cake product is packaged as individual package and multiple package. The packaging can be in air tight container. And the packaging is selected from the group consisting of paper, plastic, metal, and glass.

Examples: an example of beverage cake is made with milk and gelatin. Another example of beverage cake is made with almond milk and gelatin. Yet another example of beverage cake is made with coffee and gelatin. And yet another example of beverage cake is made with wine and gelatin. An example of sweet beverage cake is made with yogurt drink, red bean, ginseng, honey, lime juice and gelatin. Another example of sweet beverage cake is made with coconut milk, sugar, butter, beeswax, and beaten egg. An example of fruity beverage cake is made with tea, apple juice, almond, sago, and gelatin. Another example of fruity beverage cake is made with malted milk, pineapple juice, walnut, and gelatin. An example of salty beverage cake is made with soy milk, bacon, onion, salt, garlic, olive oil, gelatin, and corn starch. Another example of salty beverage cake is made with chicken soup stock, pine nut, lemongrass, basil, salt, sesame oil, and gelatin. An example of salty and spicy beverage cake is made with milk, shrimp, salt, ginger, chili, vinegar, fish liver oil, and gelatin. Another example of salty and spicy beverage cake is made with herb drink, barley, octopus, cheese, ginger, chili, olive oil, and gelatin. Many other examples can be produced; however these provide a general exemplary means for preparing the present invention.

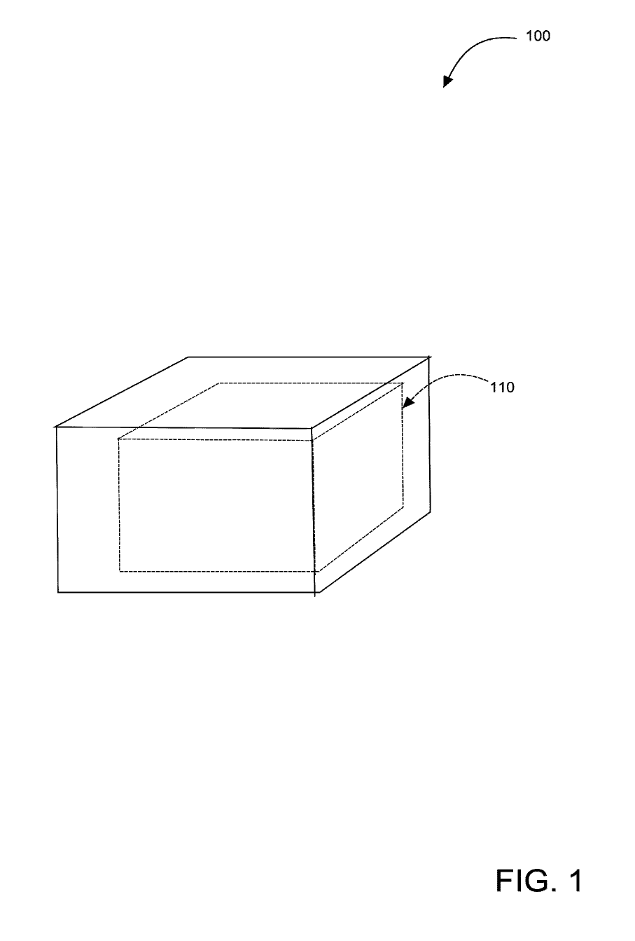

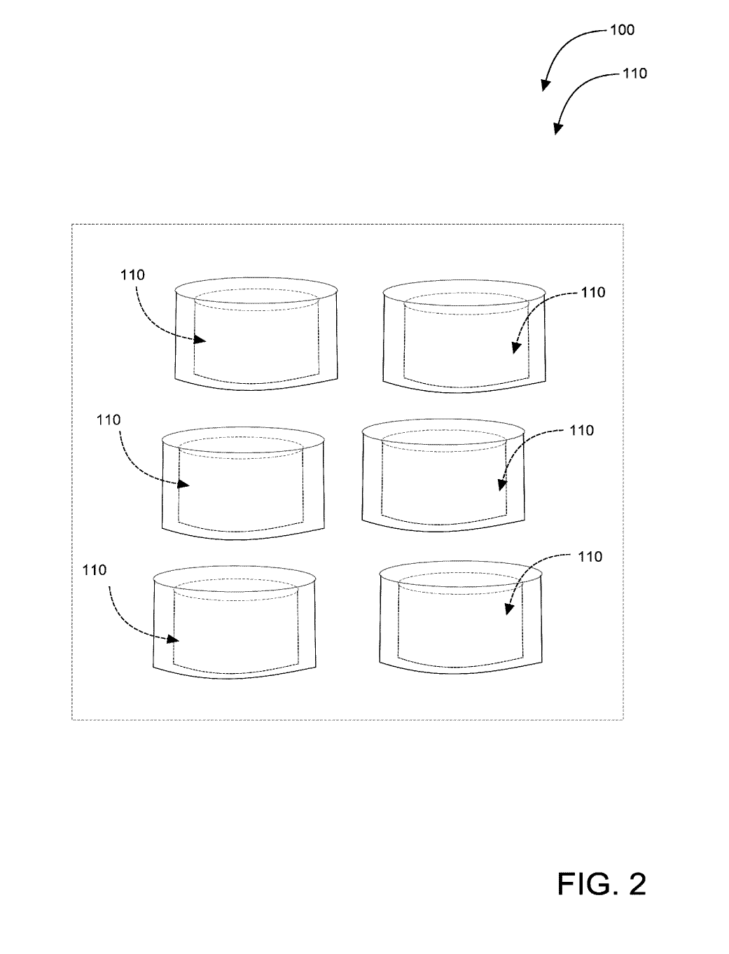

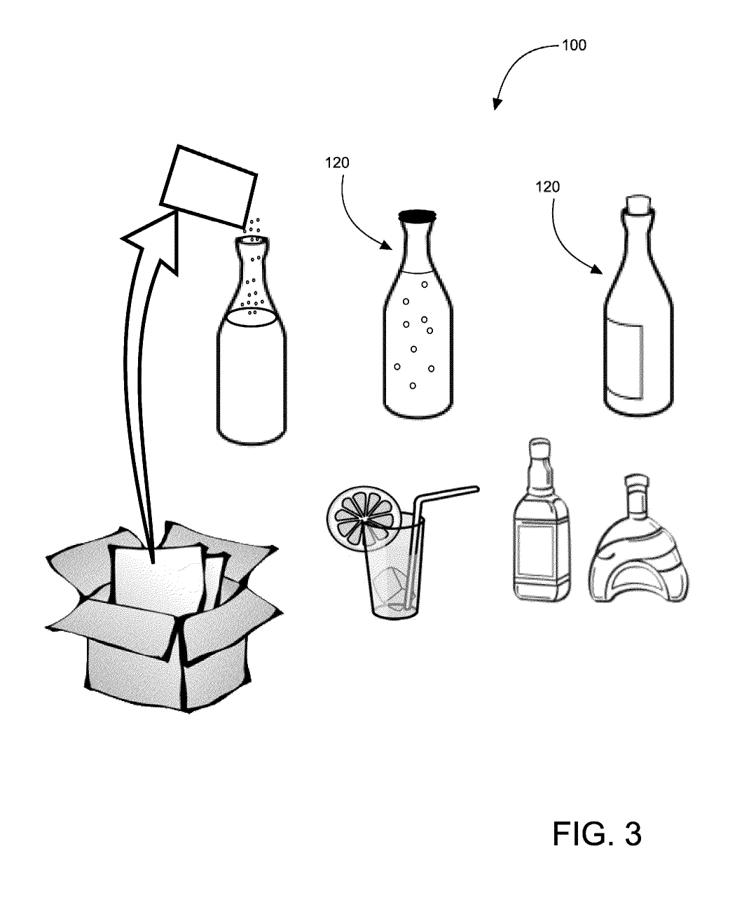

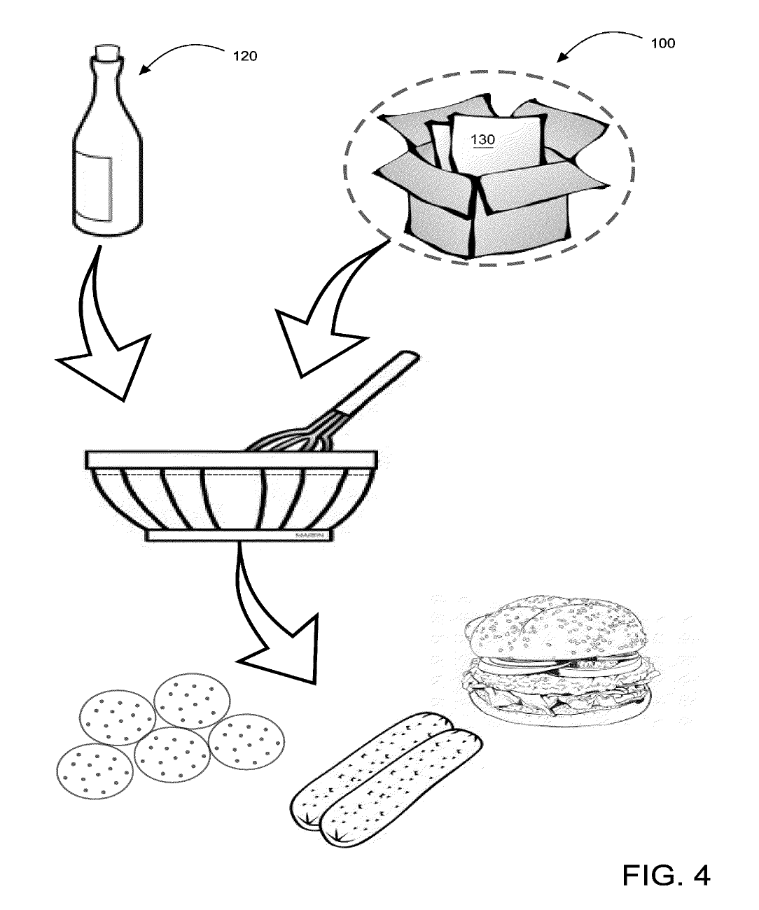

Referring generally now to the drawings by numerals of reference there is shown in FIGS. 1-4, perspective views illustrating ready to eat cold beverage cakes 110 according to embodiments of the present invention.

Ready to eat cold beverage cakes with ingredients product 100 comprises: beverage cake 110, having at least one beverage 120, and at least one binding agent 130; wherein at least one beverage 120 and at least one binding agent 130 are stirred and mixed as an admixture, and cooked to form beverage cake 110 as ready to eat cold beverage cakes with ingredients product 100.

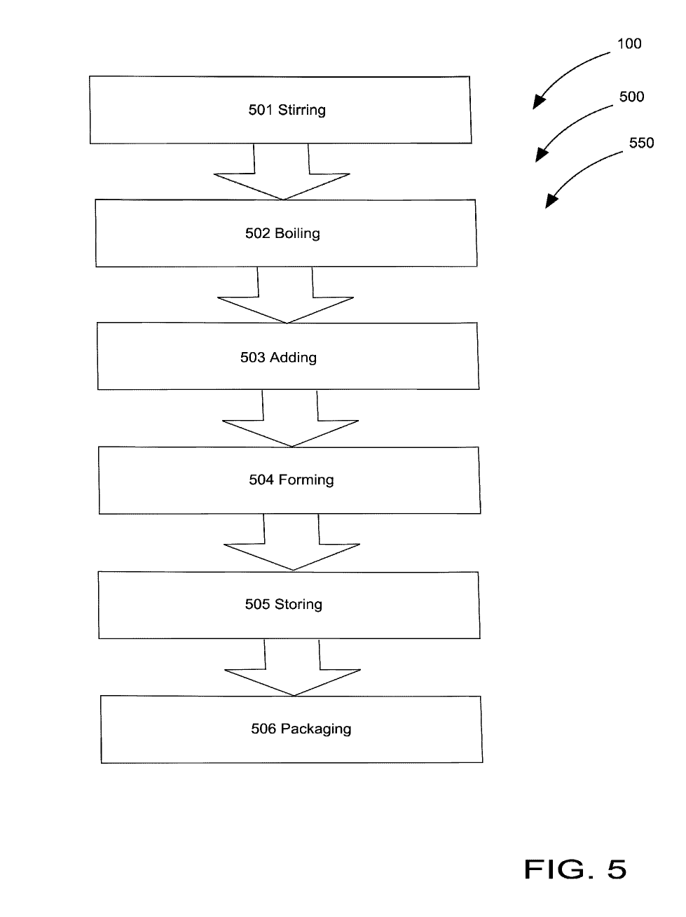

Referring now to FIG. 5, a flowchart 550 illustrating method of preparing 500 ready to eat cold beverage cakes 110 according to an embodiment of the present invention of FIGS. 1-4.

Method of preparing 500 ready to eat cold beverage cakes with ingredients 100 comprises the steps of: step one 501 stirring and mixing at least one beverage 120 with at least one binding agent 130; step two 502 boiling until the texture of the admixture is soft and smooth; step three 503 adding ingredient and flavoring as an admixture; step four 504 forming beverage cake 110; step five 505 storing beverage cake 110 in a refrigerator to congeal and solidify; and optionally step six 506 packaging to preserve freshness once solidified.

It should be noted that step 506 is an optional step and may not be implemented in all cases. Optional steps of method 500 are illustrated using dotted lines in FIG. 5 so as to distinguish them from the other steps of method 500.

It should be noted that the steps described in the method of use can be carried out in many different orders according to user preference. The use of “step of” should not be interpreted as “step for”, in the claims herein and is not intended to invoke the provisions of 35 U.S.C. §112, ¶6. Upon reading this specification, it should be appreciated that, under appropriate circumstances, considering such issues as design preference, user preferences, marketing preferences, cost, structural requirements, available materials, technological advances, etc., other methods of use arrangements such as, for example, different orders within above-mentioned list, elimination or addition of certain steps, including or excluding certain maintenance steps, etc., may be sufficient.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A ready to eat cold beverage cake product comprising:
beverage cake having
at least one beverage, and
at least one binding agent;
wherein said beverage cake is soft in texture, high in nutrition, and suitable for consumption by everybody, including animal, infant, small children, old people, and sick people; and
wherein said beverage cake can be served as breakfast, lunch, snack, and dinner.

2. The ready to eat cold beverage cake product of claim 1 wherein said beverage cake with gelatin as said at least one binding agent can be refrigerated to serve cold but not reheated because gelatin from fish and animal congeals below 15 degrees Celsius; and said beverage cake with said at least one binding agent selected from group consisting of beaten egg, egg white, and starch component can be served at room temperature, refrigerated to serve cold, and reheated to serve hot.

3. The ready to eat cold beverage cake product of claim 1 wherein said at least one beverage and said at least one binding agent are stirred and mixed as an admixture; and said beverage cake is cooked by method selected from group consisting of deep frying, boiling, steaming, roasting, frying, grilling, and baking.

4. The ready to eat cold beverage cake product of claim 1 wherein said at least one beverage is selected from group consisting of beverage, distilled water, mineral water, sports drink, tea drink, flower tea, root tea, herbal tea, mushroom tea, green tea, oolong tea, black tea, white tea, yellow tea, earl grey, darjeeling, coffee drink, soup stock, roasted cereal grain beverage, roasted nut and seed beverage, roasted bean beverage, malted drink, fruit juice, vegetable juice, fruit drink, vegetable drink, fruit skin drink, vegetable skin drink, plant drink, herb drink, soft drink, fruit punch, beer, wine, liquor, spirit, cocktail, alcoholic drink, yogurt drink, milk drink, mammal milk, plant milk, milk shake, whole milk, lowfat milk, nonfat milk, lactaid milk, chocolate milk, buttermilk, malted milk, soy milk, coconut milk, almond milk, and vanilla milk.

5. The ready to eat cold beverage cake product of claim 1 wherein said at least one binding agent is an edible binder and is selected from group consisting of protein component and starch component; said protein component having ingredient selected from group consisting of gelatin, beaten egg, and egg white; said starch component having ingredient selected from group consisting of vegetable gum, vegetable starch, and vegetable flour; said vegetable gum having ingredient selected from group consisting of agar, alginic acid, sodium alginate, carrageenan, gum arabic, gum ghatti, gum tragacanth, karaya gum, guar gum, locust bean gum, beta glucan, chicle gum, dammar gum, glucomannan gum, mastic gum, psyllium seed husk, spruce gum, tara gum, gellan gum, and xanthan gum; said vegetable starch and vegetable flour having ingredient selected from group consisting of root vegetable, cereal grain, nut, seed, bean, pectin, sago, and tapioca; said ingredient can be cooked by method selected from group consisting of mixing with water, boiling, and steaming; and said gelatin from fish and animal congeals below 15 degrees Celsius.

6. The ready to eat cold beverage cake product of claim 1 wherein said at least one binding agent selected from group consisting of beaten egg and egg white is used to create beverage egg cake; said beverage egg cake can be cooked by method selected from group consisting of deep frying, roasting, baking, steaming, boiling, grilling, and frying.

7. The ready to eat cold beverage cake product of claim 6 wherein said beverage egg cake is created in form selected from group consisting of having no outer layer; having an outer layer of casing selected from group consisting of intestine, and cellulose; comprising at least one outer coating selected from group consisting of bread crumbs, beer batter, egg batter, cornmeal batter, milk batter, yogurt batter, tea batter, coffee batter, malt batter, chocolate batter, honey batter, syrup batter, caramel batter, butterscotch batter, peanut butter batter, butter batter, margarine batter, cheese batter, cereal grain batter, nut batter, seed batter, bean batter, vegetable batter, plant batter, herb batter, fruit batter, wine batter, liquor batter, alcoholic batter, soft drink batter, soup batter, plant wax, animal wax, beeswax, carnauba wax, donut, roll, pie, pastry, waffle, crepe, pancake, pita, tortilla, bread, taco, biscuit, cookie, cake, jelly, royal jelly, cheese, cream cheese, cottage cheese, mozzarella, mascarpone, ricotta, butter, margarine, pudding, custard, candy, ice cream, gelato, sherbet, yogurt, chocolate, and fudge; having no interior filing; comprising at least one interior filing of various ingredient; having an even composition; having an uneven composition; having horizontal layers of varying composition; having vertical layers of varying composition; and having diagonal layers of varying composition.

8. The ready to eat cold beverage cake product of claim 1 wherein said beverage cake further comprising at least one flavoring ingredient causing said beverage cake to be flavored for consumption, and selected from group consisting of natural flavor, artificial flavor, fruit flavor, flower flavor, flower bud flavor, pollen flavor, leaf flavor, stem flavor, stem shoot flavor, bulb flavor, plant flavor, root flavor, herb flavor, tea flavor, nut flavor, seed flavor, bean flavor, cereal grain flavor, fruit skin flavor, vegetable oil flavor, vegetable fat flavor, vegetable skin flavor, and vegetable flavor, including hop, menthol, cordyceps, echinacea, elder, horehound, mallow, sage, thyme, cowslip, burnet, yarrow, marsh mallow, lady's mantle, speedwell, plantain, linden flowers, wild thyme, hyssop, eucalyptus, sea buckthorn, elder blossom, verbena, lemon balm, clove, coriander, cumin, basil, dill, rosemary, parsley, fennel, oregano, allspice, catnip, wormwood, musk, chili, horseradish, tumeric, mustard, curry, wasabi, mountain mint, peppermint, spearmint, wintergreen, cinnamon, licorice, rose, jasmine, chamomile, chrysanthemum, lavender, acacia, safflower, vanilla, alfafa, apple, orange, lemon, lime, grapefruit, grape, guava, plum, peach, pear, blackberry, blueberry, strawberry, cranberry, raspberry, cherry, mango, pineapple, pomegranate, papaya, watermelon, rockmelon, hamimelon, melon, honeydew, coconut, durian, passion fruit, banana, fig, apricot, mandarin, blackcurrant, starfruit, kiwi, pomelo, lychee, loquat, persimmon, tangerine, mangosteen, noni, sugar apple, mulberry, elderberry, gooseberry, lingonberry, juniper berry, jackfruit, dragonfruit, abiu, acai, acerola, ackee, cherry orange, moringa, ambarella, araza, arhat, atemoya, atherton raspberry, babaco, bacupari, bacuri, bael, barbadine, beach plum, batuan, bearberry, bignay, bilberry, bilimbi, biriba, black apple, black cherry, black raspberry, black mulberry, black sapote, blood orange, bolwarra, boquila, blue tongue, breadfruit, broad leaf bramble, buddha's hand, buffaloberry, brush cherry, burdekin plum, bush tomato, button mangosteen, caimito, calabash tree, calamondin, calamansi, camucamu, canary melon, canistel, cape gooseberry, capuli cherry, carambola, cardon, carob, cashew apple, cassabanana, cattley guava, cawesh, cedar bay cherry, ceriman, cereus peruvianus, charichuelo, chayote, che, chempedak, chenet, cherimoya, cherry ballart, chokeberry, chokecherry, chupa chupa, citron, clementine, cloudberry, cluster fig, cocky apple, coco plum, cocona, cocoplum, coffee cherry, common apple berry, conkerberry, cornelian cherry, crowberry, cupuacu, currant, curry leaf apple, custard apple, elephant apple, key apple, damson plum, date, date plum, davidson's plum, dead man's finger, desert banana, desert fig, desert lime, dodder laurel, doubah, double coconut, duku, eastern may hawthorn, emu apple, emu berry, false mastic, feijoa, fibrous satinash, finger lime, florida strangler fig, gac, galia melon, gambooge, genip, giant granadilla, otaheite gooseberry, goumi, governor's plum, montessa granadilla, yellow granadilla, greengage, grumichama, guanabana, guarana, cattley guava, pineapple guava, purple guava, strawberry guava, guavaberry, hackberry, hairless rambutan, hardy kiwi, hawthorn, hog plum, honeysuckle, horned melon, huckleberry, huito, ice cream bean, llama, lllawarra plum, imbe, jabuticaba, jambul, jatoba, jelly palm, jocote, jujube, kabosu, kaffir lime, kahikatea, kakadu lime, kakadu plum, kapok, karkalla, karonda, kei apple, kepel fruit, keule, key lime, kitembilla, korlan, kumquat, kutjera, kwai muk, lady apple, lakoocha, langsat, lanzone, lapsi, lardizabala, lemon aspen, leucaena, lillypilly, little gooseberry tree, limequat, loganberry, longan, lucuma, ma praang, mabolo, madrono, malabar plum, mamey sapote, mamoncillo, mangaba, manoao, maqui, marang, manila, mayapple, malay apple, mammee apple, maypop, medlar, melinjo, melon pear, midyim, miracle fruit, mock strawberry, purple mombin, red mombin, yellow mombin, monstera, mora de castilla, morinda, morinda citrifolia, mortiflo, mountain pepper, mountain soursop, wild plum, mundu, muntries, muscadine, muskmelon, naartjie, nageia, nance, nannyberry, naranjilla, neem, nere, nonda plum, nungu, oil palm, oregan grape, oroblanco, otaheite gooseberry, palmyra palm, pawpaw, peachpalm, peanut butter fruit, pepino, pequi, peumo, phalsa, pigeon pea, pigeon plum, pigface, pindo palm, pineapple guava, pitomba, pois doux, pommecythere, pommerac, prickly pear, prune, pulasan, pummelo, pupunha, purple apple berry, purple granadilla, quandong, queensland ebony, quince, raisin tree, rambutan, red granadilla, red mulberry, rhubarb, riberry, rimu, rollinia, rose apple, rose hip, rose leaf bramble, rose myrtle, rowan, safou, sageretia, saguaro, salak, salal, salmonberry, sandpaper fig, santol, mamey, sapodilla, fibrous satinash, saskatoonberry, saw palmetto, sea buckthorn, serviceberry, shipova, small leaf tamarind, snow berry, soncoya, servicetree, soursop, southern crabapple, spanish lime, spanish tamarind, spiny monkey orange, star apple, carambola, strawberry guava, ugni, strawberry pear, surinam cherry, sweet apple berry, sweet ganadilla, sweet lemon, sweet orange, sweet pepper, sweetsop, sycamore fig, tamarillo, tamarind, monkey tamarind, velvet tamarind, tanjong, texas persimmon, thimbleberry, totara, toyon, wampee, water apple, wax apple, wax jambu, acerola, white aspen, white mulberry, white sapote, wild grape, wild orange, wild peach, wild plum, wild mangosteen, wineberry, wolfberry, wongi, wood apple, yangmei, yellow plum, yew, youngberry, zig zag vine, ahipa, arracacha, bamboo shoot, beetroot, burdock, broadleaf arrowhead, camas, canna, carrot, cassava, celeriac, chinese artichoke, daikon, earthnut pea, elephant foot yam, ensete, galangal, ginger, ginseng, hamberg parsley, horseradish, lotus root, jerusalem artichoke, jicama, mashua, parsley root, parsnip, pignut, potato, prairie turnip, radish, rutabaga, salsify, scorzonera, skirret, swede, sweet potato, taro, ti, tigernut, tumeric, turnip, ulluco, wasabi, water caltrop, water chestnut, yalcon, yam, avocado, bell pepper, bitter melon, bitter gourd, breadfruit, chayote, cucumber, ivy gourd, eggplant, aubergine, brinjal, luffa, nopal, okra, pepper, pumpkin, squash, sweet corn, sweet pepper, tinda, tomatillo, tomato, west indian gherkin, winter melon, zucchini, courgette, artichoke, broccoli, caper, cauliflower, courgette flower, globe artichoke, squash blossom, asparagus, prussian asparagus, bamboo shoot, celery, leek, wild leek, lemongrass, rhubarb, cardoon, kurrat, galangal, amaranth, arugula, beet green, bok choy, borage, broccoli rabe, brussel sprout, cabbage, catsear, celery, celtuce, chard, chaya, chickweed, chicory, chinese mallow, chive, choi sum, chrysanthemum, collard green, common purslane, corn salad, cress, dandelion, endive, fat hen, fiddlehead, fluted pumpkin, garden rocket, golden samphire, good king henry, greater plantain, kai lan, kale, komatsuna, kuka, lagos bologi, lamb's lettuce, lamb's quarters, land cress, leek, lettuce, lizard's tail, malabar spinach, melokhia, miner's lettuce, mizuna green, mustard green, napa cabbage, new zealand spinach, orache, pak choy, paracress, pea, poke, radicchio, samphire, sculpit, stridolo, sea beet, sea kale, sierra leoni bologi, soko, sorrel, spinach, summer purslane, swiss chard, tatsoi, turnip green, watercress, water spinach, wheatgrass, winter purslane, yarrow, yao choy, cardoon, chive, florence fennel, elephant garlic, garlic, garlic chive, kohlrabi, onion, pearl onion, potato onion, spring onion, tree onion, welsh onion, quamash, scallion, shallot, rice, barley, oat, rye, corn, wheat, buckwheat, millet, sorghum, spelt, quinoa, chicory, molasses, beet root, fonio, palmer's grass, teff, triticale, cattail, chia, flax, grain amaranth, canihua, pitseed goosefoot, wattleseed, almond, cashew, chestnut, coconut, hazelnut, macadamia, peanut, pecan, pine nut, pistachio, walnut, acorn, brazil nut, gingko nut, shea nut, beechnut, candlenut, kurrajong, mongongo, palm nut, karuka, red bopple nut, hickory, jacknut, pepea nut, yellow walnut, cycad, munya nut, monkey puzzle nut, paradise nut, colocynth, filbert, mamoncillo, maya nut, ogbono nut, water caltrop, juniper, podocarp, cempedak, coffee, egusi, fox nut, jackfruit, malabar gourd, nutmeg, breadnut, betel nut, cola nut, pili nut, olive, drumstick, okra, pumpkin seed, sesame seed, sunflower seed, lotus seed, hemp seed, green bean, red bean, azuki bean, black bean, back eyed bean, white bean, yellow bean, common bean, broad bean, pinto bean, soybean, lima bean, kidney bean, pea bean, potato bean, dolichos bean, moth bean, mung bean, ricebean, runner bean, tepary bean, urad bean, velvet bean, winged bean, yardlong bean, chickpea, pigeon pea, snap pea, snow pea, lentil, lupin, tarwi, carob, guar, horse gram, cocoa bean, coffee bean, bambara groundnut, cowpea, hyacinth bean, lablab, moringa oleifera, sterculia, yam bean, alfalfa, clover, mesquite, carob, tamarind, wisteria, aonori, arame, carola, caulerpa, dabberlocks, dulse, hijiki, kombu, laver, mozuku, nori, ogonori, sea grape, sea lettuce, sea weed, wakame, truffle, mushroom, bean sprout, acai, aloe vera, arnica, ashoka, asthma weed, astragalus, barberry, belladonna, bitter leaf, bitter orange, black cohosh, blessed thistle, cat's claw, cayenne, chaparral, chasteberry, cinchona, coffee senna, comfrey, digitalis, dong quai, ephedra, european mistletoe, evening primrose, fenugreek, feverfew, flaxseed, gingko, goldenseal, hoodia, horse chestnut, horsetail, jamaica dogwood, kava, khat, konjac, kratom, kanna, licorice root, marigold, moringa oleifera, milk thistle, neem, opium poppy, purple coneflower, passion flower, red clover, syrian rue, st john's wort, saw palmetto, tea tree oil, thunder god vine, tulasi, turmeric, umckaloabo, valerian, white willow, yerba santa, ajwain, akudjura, alexanders, alkanet, alligator pepper, angelica, anise, anise hyssop, aniseed myrtle, annatto, apple mint, artemisia, asafoetida, asarabacca, avens, avocado leaf, barberry, bay leaf, bee balm, boldo, borage, black cardamom, blue fenugreek, caraway, cardamom, cassia, cayenne pepper, celery leaf, celery seed, chervil, chicory, cicely, cilantro, clary, costmary, cubeb pepper, cudweed, culantro, curry leaf, curry plant, dill seed, epazote, fenugreek, file powder, fingerroot, galangal, galingale, ganoderma, golpar, grains of paradise, grains of selim, houttuynia cordata, huacatay, Indonesian bay leaf, jimbu, kaffir lime leaves, kala zeera, kawakawa seeds, kencur, keluak, kinh gioi, kokam seed, korarima, koseret leaves, lemon myrtle, lemon ironbark, lemon verbena, leptotes bicolor, lesser calamint, lime flower, lovage, mahlab, marjoram, mastic, mountain horopito, musk mallow, nasturtium, nigella, njangsa, neem, olida, orris root, pandan flower, pandan leaf, paprika, paracress, peppermint gum leaf, perilla, pandanus amaryllifolius, quassia, ramsons, rice paddy herb, rue, saffron, salad burnet, salep, sassafras, savory, silphium, shiso, sorrel, spikenard, star anise, sumac, sweet woodruff, tarragon, voatsiperifery, water-pepper, wattleseed, wild betel, willow herb, wood avens, and woodruff; said at least one flavoring ingredient is used for said beverage cake in manner selected from group consisting of outer coating, interior filing, and mixed in; said at least one flavoring ingredient is in form selected from group consisting of skin, skinned, unskinned, unshelled, cut, uncut, extract, juice, sauce, liquid, oil, gel, jelly, wax, cream, paste, powder, thin layer, and small pieces; and said at least one flavoring ingredient can be uncooked, and cooked by method selected from group consisting of deep frying, sun drying, roasting, baking, steaming, boiling, grilling, and frying.

9. The ready to eat cold beverage cake product of claim 1 wherein said beverage cake further comprising at least one fruit flavor to make fruity flavored cake selected from group consisting of apple, orange, lemon, lime, grapefruit, grape, guava, plum, peach, pear, blackberry, blueberry, strawberry, cranberry, raspberry, cherry, mango, pineapple, pomegranate, papaya, watermelon, rockmelon, hamimelon, melon, honeydew, coconut, durian, passion fruit, banana, fig, apricot, mandarin, blackcurrant, starfruit, kiwi, pomelo, lychee, loquat, persimmon, tangerine, mangosteen, noni, sugar apple, mulberry, elderberry, gooseberry, lingonberry, juniper berry, jackfruit, dragonfruit, abiu, acai, acerola, ackee, cherry orange, moringa, ambarella, araza, arhat, atemoya, atherton raspberry, babaco, bacupari, bacuri, bael, barbadine, beach plum, batuan, bearberry, bignay, bilberry, bilimbi, biriba, black apple, black cherry, black raspberry, black mulberry, black sapote, blood orange, bolwarra, boquila, blue tongue, breadfruit, broad leaf bramble, buddha's hand, buffaloberry, brush cherry, burdekin plum, bush tomato, button mangosteen, caimito, calabash tree, calamondin, calamansi, camucamu, canary melon, canistel, cape gooseberry, capuli cherry, carambola, cardon, carob, cashew apple, cassabanana, cattley guava, cawesh, cedar bay cherry, ceriman, cereus peruvianus, charichuelo, chayote, che, chempedak, chenet, cherimoya, cherry ballart, chokeberry, chokecherry, chupa chupa, citron, clementine, cloudberry, cluster fig, cocky apple, coco plum, cocona, cocoplum, coffee cherry, common apple berry, conkerberry, cornelian cherry, crowberry, cupuacu, currant, curry leaf apple, custard apple, elephant apple, key apple, damson plum, date, date plum, davidson's plum, dead man's finger, desert banana, desert fig, desert lime, dodder laurel, doubah, double coconut, duku, eastern may hawthorn, emu apple, emu berry, false mastic, feijoa, fibrous satinash, finger lime, florida strangler fig, gac, galia melon, gambooge, genip, giant granadilla, otaheite gooseberry, goumi, governor's plum, montessa granadilla, yellow granadilla, greengage, grumichama, guanabana, guarana, cattley guava, pineapple guava, purple guava, strawberry guava, guavaberry, hackberry, hairless rambutan, hardy kiwi, hawthorn, hog plum, honeysuckle, horned melon, huckleberry, huito, ice cream bean, llama, lllawarra plum, imbe, jabuticaba, jambul, jatoba, jelly palm, jocote, jujube, kabosu, kaffir lime, kahikatea, kakadu lime, kakadu plum, kapok, karkalla, karonda, kei apple, kepel fruit, keule, key lime, kitembilla, korlan, kumquat, kutjera, kwai muk, lady apple, lakoocha, langsat, lanzone, lapsi, lardizabala, lemon aspen, leucaena, lillypilly, little gooseberry tree, limequat, loganberry, longan, lucuma, ma praang, mabolo, madrono, malabar plum, mamey sapote, mamoncillo, mangaba, manoao, maqui, marang, marula, mayapple, malay apple, mammee apple, maypop, medlar, melinjo, melon pear, midyim, miracle fruit, mock strawberry, purple mombin, red mombin, yellow mombin, monstera, mora de castilla, morinda, morinda citrifolia, mortiflo, mountain pepper, mountain soursop, wild plum, mundu, muntries, muscadine, muskmelon, naartjie, nageia, nance, nannyberry, naranjilla, neem, nere, nonda plum, nungu, oil palm, oregan grape, oroblanco, otaheite gooseberry, palmyra palm, pawpaw, peachpalm, peanut butter fruit, pepino, pequi, peumo, phalsa, pigeon pea, pigeon plum, pigface, pindo palm, pineapple guava, pitomba, pois doux, pommecythere, pommerac, prickly pear, prune, pulasan, pummelo, pupunha, purple apple berry, purple granadilla, quandong, queensland ebony, quince, raisin tree, rambutan, red granadilla, red mulberry, rhubarb, riberry, rimu, rollinia, rose apple, rose hip, rose leaf bramble, rose myrtle, rowan, safou, sageretia, saguaro, salak, salal, salmonberry, sandpaper fig, santol, mamey, sapodilla, fibrous satinash, saskatoonberry, saw palmetto, sea buckthorn, serviceberry, shipova, small leaf tamarind, snow berry, soncoya, servicetree, soursop, southern crabapple, spanish lime, spanish tamarind, spiny monkey orange, star apple, carambola, strawberry guava, ugni, strawberry pear, surinam cherry, sweet apple berry, sweet ganadilla, sweet lemon, sweet orange, sweet pepper, sweetsop, sycamore fig, tamarillo, tamarind, monkey tamarind, velvet tamarind, tanjong, texas persimmon, thimbleberry, totara, toyon, wampee, water apple, wax apple, wax jambu, acerola, white aspen, white mulberry, white sapote, wild grape, wild orange, wild peach, wild plum, wild mangosteen, wineberry, wolfberry, wongi, wood apple, yangmei, yellow plum, yew, youngberry, and zig zag vine; said at least one fruit flavor is used for said beverage cake in manner selected from group consisting of outer coating, interior filing, and mixed in; said at least one fruit flavor is in form selected from group consisting of skin, skinned, unskinned, unshelled, cut, uncut, extract, juice, sauce, liquid, oil, gel, jelly, wax, cream, paste, powder, thin layer, and small pieces; and said at least one fruit flavor can be uncooked, and cooked by method selected from group consisting of deep frying, sun drying, roasting, baking, steaming, boiling, grilling, and frying.

10. The ready to eat cold beverage cake product of claim 1 wherein said beverage cake further comprising at least one other flavoring ingredient selected from group consisting of natural flavor, artificial flavor, artificial sweetener, salt, vinegar, sugar, syrup, honey, fudge, chocolate, royal jelly, jelly, pudding, custard, ice cream, sherbet, gelato, cake, biscuit, cookie, crepe, pancake, donut, roll, pie, pastry, waffle, pita, tortilla, bread, taco, candy, marshmallow, caramel, butterscotch, raisin, apple sauce, vegetable shortening, vegetable oil, vegetable fat, fish oil, fish liver oil, plant wax, animal wax, beeswax, carnauba wax, cream, sour cream, yogurt, butter, margarine, peanut butter, cheese, cream cheese, cottage cheese, mozzarella, mascarpone, ricotta, egg, glycerol, suet, lard, and malt; said at least one other flavoring ingredient is used for said beverage cake in manner selected from group consisting of outer coating, interior filing, and mixed in; and said at least one other flavoring ingredient is in form selected from group consisting of extract, juice, sauce, liquid, oil, gel, jelly, wax, cream, paste, powder, thin layer, and small pieces.

11. The ready to eat cold beverage cake product of claim 1 wherein said beverage cake further comprising at least one nutritional ingredient which is essential nutrient selected from group consisting of vitamin, dietary mineral, amino acid, and fatty acid; said vitamin includes vitamin A, vitamin B1, vitamin B2, vitamin B3, vitamin B5, vitamin B6, vitamin B7, vitamin B9, vitamin B12, vitamin C, vitamin D, vitamin E, and vitamin K; said dietary mineral includes calcium, phosphorus, potassium, sulfur, sodium, chlorine, magnesium, iron, cobalt, copper, zinc, molybdenum, iodine, selenium, manganese, nickle, chromium, fluorine, boron, and strontium; said amino acid includes histidine, isoleucine, leucine, lysine, methionine, cysteine, phenylalanine, tyrosine, threonine, tryptophan, and valine; said fatty acid includes alpha-linoleic acid, and linoleic acid; said at least one nutritional ingredient is used for said beverage cake in manner selected from group consisting of outer coating, interior filing, and mixed in; and said at least one nutritional ingredient is in form selected from group consisting of extract, juice, sauce, liquid, oil, gel, jelly, wax, cream, paste, powder, thin layer, and small pieces.

12. The ready to eat cold beverage cake product of claim 1 wherein said beverage cake further comprising at least one health ingredient which includes medication and other health ingredient; said medication is mixed into said beverage cake for ease of consumption; said other health ingredient is selected from group consisting of electrolyte, lactase, yeast, propolis, and quinine; and said at least one health ingredient is in form selected from group consisting of extract, juice, sauce, liquid, oil, gel, jelly, wax, cream, paste, powder, thin layer, and small pieces.

13. The ready to eat cold beverage cake product of claim 1 wherein said beverage cake further comprising at least one food additive selected from group consisting of acid, acidity regulator, alcohol, anticaking agent, antifoaming agent, antioxidant, bulking agent, coagulation agent, food coloring, color retention agent, emulsifier, flavor enhancer, flour treatment agent, gelling agent, glazing agent, humectant, leavening agent, tracer gas, preservative, stabilizer, sweetener, and thickener.

14. The ready to eat cold beverage cake product of claim 1 wherein said beverage cake further comprising at least one topping selected from group consisting of chocolate, caramel, butterscotch, peanut butter, butter, margarine, vegetable shortening, vegetable oil, vegetable fat, fish oil, fish liver oil, plant wax, animal wax, beeswax, carnauba wax, sugar, syrup, honey, royal jelly, apple sauce, raisin, fruit, vegetable, fruit skin, vegetable skin, herb, cereal grain, nut, seed, bean, cheese, cream cheese, cottage cheese, mozzarella, mascarpone, ricotta, jelly, pudding, custard, ice cream, yogurt, sherbet, gelato, cake, biscuit, cookie, crepe, pancake, donut, roll, pie, pastry, waffle, pita, tortilla, bread, taco, candy, marshmallow, sour cream, cream, malt, egg, meat, and seafood; said topping is in form selected from group consisting of extract, juice, sauce, liquid, oil, gel, jelly, wax, cream, paste, powder, thin layer, and small pieces; and said topping can be uncooked, and cooked by method selected from group consisting of deep frying, sun drying, roasting, baking, steaming, boiling, grilling, and frying.

15. The ready to eat cold beverage cake product of claim 1 wherein said beverage cake further comprising at least one salt source having a combination of ingredients selected from group consisting of seafood, meat, and cheese, including seafood and meat, seafood and cheese, meat and cheese, seafood and meat and cheese; said seafood is selected from group consisting of squid, cuttle fish, shrimp, crab, crayfish, octopus, clam, oyster, scallop, lobster, abalone, conch, jellyfish, prawn, hill, mussel, sea cucumber, sea urchin, eel, snail, shrimp roe, crab roe, lobster roe, fish roe, and fish; said meat is selected from group consisting of chicken, duck, goose, turkey, quail, rabbit, sheep, goat, cow, pig, deer, bison, buffalo, crocodile, frog, turtle, snail, worm, bacon, sausage, animal tissue, animal organ, animal tongue, animal tail, animal feet, animal liver, animal stomach, animal intestine, animal brain, animal heart, animal skin, animal tendon, animal fat, animal bone marrow, and animal blood; said at least one salt source is used for said beverage cake in manner selected from group consisting of outer coating, interior filing, and mixed in; said at least one salt source is selected from group consisting of cut, uncut, slice into thin layer, cut into small pieces, and grind into small particles; and said at least one salt source can be cooked by method selected from group consisting of deep frying, roasting, baking, steaming, boiling, grilling, and frying.

16. The ready to eat cold beverage cake product of claim 15 wherein said at least one salt source having a combination of ingredients is grinded to create beverage plus salt source cake; said beverage plus salt source cake is prepared with a small amount of said at least one liquid ingredient; said beverage plus salt source cake is having said at least one binding agent selected from group consisting of beaten egg, egg white, and starch component; and said beverage plus salt source cake is cooked by method selected from group consisting of deep frying, roasting, baking, boiling, steaming, grilling, and frying.

17. The ready to eat cold beverage cake product of claim 16 wherein said beverage plus salt source cake is created in form selected from group consisting of having no outer layer; having an outer layer of casing selected from group consisting of intestine, and cellulose; comprising at least one outer coating selected from group consisting of bread crumbs, beer batter, egg batter, cornmeal batter, milk batter, yogurt batter, tea batter, coffee batter, malt batter, chocolate batter, honey batter, syrup batter, caramel batter, butterscotch batter, peanut butter batter, butter batter, margarine batter, cheese batter, cereal grain batter, nut batter, seed batter, bean batter, vegetable batter, plant batter, herb batter, fruit batter, wine batter, liquor batter, alcoholic batter, soft drink batter, soup batter, plant wax, animal wax, beeswax, carnauba wax, donut, roll, pie, pastry, waffle, crepe, pancake, pita, tortilla, bread, taco, biscuit, cookie, cake, jelly, royal jelly, cheese, cream cheese, cottage cheese, mozzarella, mascarpone, ricotta, butter, margarine, pudding, custard, candy, ice cream, gelato, sherbet, yogurt, chocolate, and fudge; having no interior filing; comprising at least one interior filing of various ingredient; having an even composition; having an uneven composition; having horizontal layers of varying composition; having vertical layers of varying composition; and having diagonal layers of varying composition.

18. The ready to eat cold beverage cake product of claim 1 wherein said beverage cake further comprising at least one seafood flavor selected from group consisting of squid, cuttle fish, shrimp, crab, crayfish, octopus, clam, oyster, scallop, lobster, abalone, conch, jellyfish, prawn, krill, mussel, sea cucumber, sea urchin, eel, snail, shrimp roe, crab roe, lobster roe, fish roe, and fish; said at least one seafood flavor is used for said beverage cake in manner selected from group consisting of outer coating, interior filing, and mixed in; said at least one seafood flavor is selected from group consisting of cut, uncut, slice into thin layer, cut into small pieces, and grind into small particles; and said at least one seafood flavor can be cooked by method selected from group consisting of deep frying, roasting, baking, steaming, boiling, grilling, and frying.

19. The ready to eat cold beverage cake product of claim 18 wherein said at least one seafood is grinded and used as ingredient to create beverage seafood cake; said beverage seafood cake is prepared with a small amount of said at least one liquid ingredient; said beverage seafood cake is having said at least one binding agent selected from group consisting of beaten egg, egg white, and starch component; and said beverage seafood cake is cooked by method selected from group consisting of roasting, baking, boiling, steaming, grilling, and frying.

20. The ready to eat cold beverage cake product of claim 19 wherein said beverage seafood cake is created in form selected from group consisting of having no outer layer; having an outer layer of casing selected from group consisting of intestine, and cellulose; comprising at least one outer coating selected from group consisting of bread crumbs, beer batter, egg batter, cornmeal batter, milk batter, yogurt batter, tea batter, coffee batter, malt batter, chocolate batter, honey batter, syrup batter, caramel batter, butterscotch batter, peanut butter batter, butter batter, margarine batter, cheese batter, cereal grain batter, nut batter, seed batter, bean batter, vegetable batter, plant batter, herb batter, fruit batter, wine batter, liquor batter, alcoholic batter, soft drink batter, soup batter, plant wax, animal wax, beeswax, carnauba wax, donut, roll, pie, pastry, waffle, crepe, pancake, pita, tortilla, bread, taco, biscuit, cookie, cake, jelly, royal jelly, cheese, cream cheese, cottage cheese, mozzarella, mascarpone, ricotta, butter, margarine, pudding, custard, candy, ice cream, gelato, sherbet, yogurt, chocolate, and fudge; having no interior filing; comprising at least one interior filing of various ingredient; having an even composition; having an uneven composition; having horizontal layers of varying composition; having vertical layers of varying composition; and having diagonal layers of varying composition.

21. The ready to eat cold beverage cake product of claim 1 wherein said beverage cake further comprising at least one meat flavor selected from group consisting of chicken, duck, goose, turkey, quail, rabbit, sheep, goat, cow, pig, deer, bison, buffalo, crocodile, frog, turtle, snail, worm, bacon, sausage, animal tissue, animal organ, animal tongue, animal tail, animal feet, animal liver, animal stomach, animal intestine, animal brain, animal heart, animal skin, animal tendon, animal fat, animal bone marrow, and animal blood; said at least one meat flavor is used for said beverage cake in manner selected from group consisting of outer coating, interior filing, and mixed in; said at least one meat flavor is selected from group consisting of cut, uncut, slice into thin layer, cut into small pieces, and grind into small particles; and said at least one meat flavor can be cooked by method selected from group consisting of deep frying, roasting, baking, steaming, boiling, grilling, and frying.

22. The ready to eat cold beverage cake product of claim 21 wherein said at least one meat is grinded and used as ingredient to create beverage meat cake; said beverage meat cake is prepared with a small amount of said at least one liquid ingredient; said beverage meat cake is having said at least one binding agent selected from group consisting of egg, egg white, and starch component; and said beverage meat cake is cooked by method selected from group consisting of deep frying, roasting, baking, boiling, steaming, grilling, and frying.

23. The ready to eat cold beverage cake product of claim 22 wherein said beverage meat cake is created in form selected from group consisting of having no outer layer; having an outer layer of casing selected from group consisting of intestine, and cellulose; comprising at least one outer coating selected from group consisting of bread crumbs, beer batter, egg batter, cornmeal batter, milk batter, yogurt batter, tea batter, coffee batter, malt batter, chocolate batter, honey batter, syrup batter, caramel batter, butterscotch batter, peanut butter batter, butter batter, margarine batter, cheese batter, cereal grain batter, nut batter, seed batter, bean batter, vegetable batter, plant batter, herb batter, fruit batter, wine batter, liquor batter, alcoholic batter, soft drink batter, soup batter, plant wax, animal wax, beeswax, carnauba wax, donut, roll, pie, pastry, waffle, crepe, pancake, pita, tortilla, bread, taco, biscuit, cookie, cake, jelly, royal jelly, cheese, cream cheese, cottage cheese, mozzarella, mascarpone, ricotta, butter, margarine, pudding, custard, candy, ice cream, gelato, sherbet, yogurt, chocolate, and fudge; having no interior filing; comprising at least one interior filing of various ingredient; having an even composition; having an uneven composition; having horizontal layers of varying composition; having vertical layers of varying composition; and having diagonal layers of varying composition.

24. The ready to eat cold beverage cake product of claim 1 wherein said beverage cake further comprising at least one vegetable flavor selected from group consisting of root vegetable, fruit vegetable, flower bud, flower, stem shoot, stem, leaf, bulb, cereal grain, nut, seed, bean, sea vegetable, and other types of vegetable, including ahipa, arracacha, bamboo shoot, beetroot, burdock, broadleaf arrowhead, camas, canna, carrot, cassava, celeriac, chinese artichoke, daikon, earthnut pea, elephant foot yam, ensete, galangal, ginger, ginseng, hamberg parsley, horseradish, lotus root, jerusalem artichoke, jicama, mashua, parsley root, parsnip, pignut, potato, prairie turnip, radish, rutabaga, salsify, scorzonera, skirret, swede, sweet potato, taro, ti, tigernut, tumeric, turnip, ulluco, wasabi, water caltrop, water chestnut, yalcon, yam, avocado, bell pepper, bitter melon, bitter gourd, breadfruit, chayote, cucumber, ivy gourd, eggplant, aubergine, brinjal, luffa, nopal, okra, pepper, pumpkin, squash, sweet corn, sweet pepper, tinda, tomatillo, tomato, west indian gherkin, winter melon, zucchini, courgette, artichoke, broccoli, caper, cauliflower, courgette flower, globe artichoke, squash blossom, asparagus, prussian asparagus, bamboo shoot, celery, leek, wild leek, lemongrass, rhubarb, cardoon, kurrat, galangal, amaranth, arugula, beet green, bok choy, borage, broccoli rabe, brussel sprout, cabbage, catsear, celery, celtuce, chard, chaya, chickweed, chicory, chinese mallow, chive, choi sum, chrysanthemum, collard green, common purslane, corn salad, cress, dandelion, endive, fat hen, fiddlehead, fluted pumpkin, garden rocket, golden samphire, good king henry, greater plantain, kai lan, kale, komatsuna, kuka, lagos bologi, lamb's lettuce, lamb's quarters, land cress, leek, lettuce, lizard's tail, malabar spinach, melokhia, miner's lettuce, mizuna green, mustard green, napa cabbage, new zealand spinach, orache, pak choy, paracress, pea, poke, radicchio, samphire, sculpit, stridolo, sea beet, sea kale, sierra leoni bologi, soko, sorrel, spinach, summer purslane, swiss chard, tatsoi, turnip green, watercress, water spinach, wheatgrass, winter purslane, yarrow, yao choy, cardoon, chive, florence fennel, elephant garlic, garlic, garlic chive, kohlrabi, onion, pearl onion, potato onion, spring onion, tree onion, welsh onion, quamash, scallion, shallot, rice, barley, oat, rye, corn, wheat, buckwheat, millet, sorghum, spelt, quinoa, chicory, molasses, beet root, fonio, palmer's grass, teff, triticale, cattail, chia, flax, grain amaranth, canihua, pitseed goosefoot, wattleseed, almond, cashew, chestnut, coconut, hazelnut, macadamia, peanut, pecan, pine nut, pistachio, walnut, acorn, brazil nut, gingko nut, shea nut, beechnut, candlenut, kurrajong, mongongo, palm nut, karuka, red bopple nut, hickory, jacknut, pepea nut, yellow walnut, cycad, munya nut, monkey puzzle nut, paradise nut, colocynth, filbert, mamoncillo, maya nut, ogbono nut, water caltrop, juniper, podocarp, cempedak, coffee, egusi, fox nut, jackfruit, malabar gourd, nutmeg, breadnut, betel nut, cola nut, pili nut, olive, drumstick, okra, pumpkin seed, sesame seed, sunflower seed, lotus seed, hemp seed, green bean, red bean, azuki bean, black bean, back eyed bean, white bean, yellow bean, common bean, broad bean, pinto bean, soybean, lima bean, kidney bean, pea bean, potato bean, dolichos bean, moth bean, mung bean, ricebean, runner bean, tepary bean, urad bean, velvet bean, winged bean, yardlong bean, chickpea, pigeon pea, snap pea, snow pea, lentil, lupin, tarwi, carob, guar, horse gram, cocoa bean, coffee bean, bambara groundnut, cowpea, hyacinth bean, lablab, moringa oleifera, sterculia, yam bean, alfalfa, clover, mesquite, carob, tamarind, wisteria, aonori, arame, carola, caulerpa, dabberlocks, dulse, hijiki, kombu, laver, mozuku, nori, ogonori, sea grape, sea lettuce, sea weed, wakame, truffle, mushroom, and bean sprout; said at least one vegetable flavor is used for said beverage cake in manner selected from group consisting of outer coating, interior filing, and mixed in; said at least one vegetable flavor is in form selected from group consisting of skin, skinned, unskinned, unshelled, cut, uncut, extract, juice, sauce, liquid, oil, gel, jelly, wax, cream, paste, powder, thin layer, and small pieces; and said at least one vegetable flavor can be uncooked, and cooked by method selected from group consisting of deep frying, sun drying, roasting, baking, steaming, boiling, grilling, and frying.

25. The ready to eat cold beverage cake product of claim 1 wherein said beverage cake is created in form selected from group consisting of having no outer layer; having an outer layer of casing selected from group consisting of intestine, and cellulose; comprising at least one outer coating selected from group consisting of bread crumbs, beer batter, egg batter, cornmeal batter, milk batter, yogurt batter, tea batter, coffee batter, malt batter, chocolate batter, honey batter, syrup batter, caramel batter, butterscotch batter, peanut butter batter, butter batter, margarine batter, cheese batter, cereal grain batter, nut batter, seed batter, bean batter, vegetable batter, plant batter, herb batter, fruit batter, wine batter, liquor batter, alcoholic batter, soft drink batter, soup batter, plant wax, animal wax, beeswax, carnauba wax, donut, roll, pie, pastry, waffle, crepe, pancake, pita, tortilla, bread, taco, biscuit, cookie, cake, jelly, royal jelly, cheese, cream cheese, cottage cheese, mozzarella, mascarpone, ricotta, butter, margarine, pudding, custard, candy, ice cream, gelato, sherbet, yogurt, chocolate, and fudge; having no interior filing; comprising at least one interior filing of various ingredient; having an even composition; having an uneven composition; having horizontal layers of varying composition; having vertical layers of varying composition; and having diagonal layers of varying composition.

26. The ready to eat cold beverage cake product of claim 1 wherein said beverage cake having a variety of shapes and sizes is selected from group consisting of sphere, disc, tube, cube, cylinder, cone, triangle, hexagon, octagon, rectangle, and oval; said beverage cake having a variety of shapes and sizes selected from group consisting of cubical shape with each side of about 1 to 10 inches, spherical shape with diameter of about 1 to 100 millimeters, disc shape with diameter of about 3 to 5 inches and thickness of about ½ to 2 inches, and tube shape with diameter of about ½ to 2 inches and length of about 1 to 10 inches.

27. The ready to eat cold beverage cake product of claim 26 wherein said spherical shape having diameter of about 1 to 10 millimeters is created as beverage bubble; and said beverage bubble is cooked by method selected from group consisting of deep frying, roasting, baking, steaming, boiling, grilling, and frying.

28. The ready to eat cold beverage cake product of claim 26 wherein said spherical shape having diameter of about 10 to 40 millimeters is created as beverage ball; and said beverage ball is cooked by method selected from group consisting of deep frying, roasting, baking, steaming, boiling, grilling, and frying.

29. The ready to eat cold beverage cake product of claim 28 wherein said beverage ball is created in form selected from group consisting of spherical shape without an outer layer; spherical shape with an outer layer of casing selected from group consisting of intestine, and cellulose; spherical shape comprising at least one outer coating selected from group consisting of bread crumbs, beer batter, egg batter, cornmeal batter, milk batter, yogurt batter, tea batter, coffee batter, malt batter, chocolate batter, honey batter, syrup batter, caramel batter, butterscotch batter, peanut butter batter, butter batter, margarine batter, cheese batter, cereal grain batter, nut batter, seed batter, bean batter, vegetable batter, plant batter, herb batter, fruit batter, wine batter, liquor batter, alcoholic batter, soft drink batter, soup batter, plant wax, animal wax, beeswax, carnauba wax, donut, roll, pie, pastry, waffle, crepe, pancake, pita, tortilla, bread, taco, biscuit, cookie, cake, jelly, royal jelly, cheese, cream cheese, cottage cheese, mozzarella, mascarpone, ricotta, butter, margarine, pudding, custard, candy, ice cream, gelato, sherbet, yogurt, chocolate, and fudge; having no interior filing; comprising at least one interior filing of various ingredient; having an even composition; having an uneven composition; having horizontal layers of varying composition; having vertical layers of varying composition; and having diagonal layers of varying composition.

30. The ready to eat cold beverage cake product of claim 26 wherein said spherical shape and said variety of shapes and sizes are mixable in drink, food, and condiment; and are mixable in ingredient selected from group consisting of yogurt, ice cream, cake, rice, mashed potato, pasta, noodle, salad dressing, sauce, spread, beverage, distilled water, mineral water, sports drink, tea drink, flower tea, root tea, herbal tea, mushroom tea, green tea, oolong tea, black tea, white tea, yellow tea, earl grey, darjeeling, coffee drink, soup stock, roasted cereal grain beverage, roasted nut and seed beverage, roasted bean beverage, malted drink, fruit juice, vegetable juice, fruit drink, vegetable drink, fruit skin drink, vegetable skin drink, plant drink, herb drink, soft drink, fruit punch, beer, wine, liquor, spirit, cocktail, alcoholic drink, yogurt drink, milk drink, mammal milk, plant milk, milk shake, whole milk, lowfat milk, nonfat milk, lactaid milk, chocolate milk, buttermilk, malted milk, soy milk, coconut milk, almond milk, and vanilla milk.

31. The ready to eat cold beverage cake product of claim 26 wherein said disc shape having diameter of about 1½ to 5 inches and thickness of about ⅓ to 2 inches is created as beverage patty; said beverage patty is used to make burger; and said beverage patty is cooked by method selected from group consisting of deep frying, roasting, baking, steaming, boiling, grilling, and frying.

32. The ready to eat cold beverage cake product of claim 31 wherein said beverage patty is created in form selected from group consisting of disc shape without an outer layer; disc shape with an outer layer of casing selected from group consisting of intestine, and cellulose; disc shape comprising at least one outer coating selected from group consisting of bread crumbs, beer batter, egg batter, cornmeal batter, milk batter, yogurt batter, tea batter, coffee batter, malt batter, chocolate batter, honey batter, syrup batter, caramel batter, butterscotch batter, peanut butter batter, butter batter, margarine batter, cheese batter, cereal grain batter, nut batter, seed batter, bean batter, vegetable batter, plant batter, herb batter, fruit batter, wine batter, liquor batter, alcoholic batter, soft drink batter, soup batter, plant wax, animal wax, beeswax, carnauba wax, donut, roll, pie, pastry, waffle, crepe, pancake, pita, tortilla, bread, taco, biscuit, cookie, cake, jelly, royal jelly, cheese, cream cheese, cottage cheese, mozzarella, mascarpone, ricotta, butter, margarine, pudding, custard, candy, ice cream, gelato, sherbet, yogurt, chocolate, and fudge; having no interior filing; comprising at least one interior filing of various ingredient; having an even composition; having an uneven composition; having horizontal layers of varying composition; having vertical layers of varying composition; and having diagonal layers of varying composition.

33. The ready to eat cold beverage cake product of claim 26 wherein said tube shape having diameter of about ½ to 2 inches and length of about 1 to 10 inches is created as beverage sausage; said beverage sausage is used to make hot dog; and said beverage sausage is cooked by method selected from group consisting of deep frying, roasting, baking, steaming, boiling, grilling, and frying.

34. The ready to eat cold beverage cake product of claim 33 wherein said beverage sausage is created in form selected from group consisting of tube shape without an outer layer; tube shape with an outer layer of casing selected from group consisting of intestine, and cellulose; tube shape comprising at least one outer coating selected from group consisting of bread crumbs, beer batter, egg batter, cornmeal batter, milk batter, yogurt batter, tea batter, coffee batter, malt batter, chocolate batter, honey batter, syrup batter, caramel batter, butterscotch batter, peanut butter batter, butter batter, margarine batter, cheese batter, cereal grain batter, nut batter, seed batter, bean batter, vegetable batter, plant batter, herb batter, fruit batter, wine batter, liquor batter, alcoholic batter, soft drink batter, soup batter, plant wax, animal wax, beeswax, carnauba wax, donut, roll, pie, pastry, waffle, crepe, pancake, pita, tortilla, bread, taco, biscuit, cookie, cake, jelly, royal jelly, cheese, cream cheese, cottage cheese, mozzarella, mascarpone, ricotta, butter, margarine, pudding, custard, candy, ice cream, gelato, sherbet, yogurt, chocolate, and fudge; having no interior filing; comprising at least one interior filing of various ingredient; having an even composition; having an uneven composition; having horizontal layers of varying composition; having vertical layers of varying composition; and having diagonal layers of varying composition.

35. The ready to eat cold beverage cake product of claim 1 having packaging as individual package and multiple package; said packaging is in air tight container; and said packaging is selected from group consisting of paper, plastic, metal, and glass.

36. A method of preparing said ready to eat cold beverage cake product of claim 1 comprising the steps of: stirring and mixing said at least one beverage with said at least one binding agent; boiling until texture of admixture is soft and smooth; adding ingredient and flavoring as an admixture; forming said beverage cake; storing said beverage cake in a refrigerator to congeal and solidify; and packaging to preserve freshness once solidified.
